(19) United States
(12) Patent Application Publication
(10) Pub. No.: US 2015/0064107 A1
(43) Pub. Date: Mar. 5, 2015

(54) IMAGING AGENT

(71) Applicant: King's College London, London (GB)

(72) Inventors: Xianjin Cui, Brighton (GB); Philip Blower, Petersfield (GB); Mark A. Green, Necton (GB)

(73) Assignee: King's College London, London (GB)

(21) Appl. No.: 14/471,017

(22) Filed: Aug. 28, 2014

Related U.S. Application Data
(60) Provisional application No. 61/873,670, filed on Sep. 4, 2013.

(51) Int. Cl. A61K 49/18; A61K 51/02; A61K 51/12; A61K 49/00; A61K 49/08

(52) U.S. Cl. CPC A61K 49/1866; A61K 49/005; A61K 49/00; A61K 49/0093; A61K 49/08; A61K 49/1827; A61K 51/1244; A61K 51/025
USPC 424/1.29; 424/9.32; 424/9.323; 424/9.6; 424/9.1; 427/2.12

(57) ABSTRACT

The invention relates to imaging agents, and in particular to multi-modal nanoparticle (NPIA) imaging agents offering magnetic, radionuclide and fluorescent imaging capabilities to exploit the complementary advantages of magnetic resonance imaging (MRI), positron emission tomography (PET), single-photon emission computed tomography (SPECT) and optical imaging (OI). The invention extends to these new types of agents per se, and to uses of such agents in various biomedical applications, such as in therapy and in diagnosis.

### RELATED APPLICATIONS

This application claims priority under 35 U.S.C. §119(e) to U.S. Provisional Application Ser. No. 61/873,670, entitled “IMAGING AGENT” filed on Sep. 4, 2013, which is herein incorporated by reference in its entirety.

The invention relates to imaging agents, and in particular to multi-modal nanoparticle (NPIA) imaging agents offering magnetic, radionuclide and fluorescent imaging capabilities to exploit the complementary advantages of magnetic resonance imaging (MRI), positron emission tomography (PET), single-photon emission computed tomography (SPECT) and optical imaging (OI). The invention extends to these new types of agents per se, and to uses of such agents in various biomedical applications, such as in therapy and in diagnosis.

The potential clinical and biomedical application of synergistic combinations of MRI with other biomedical imaging modalities, especially PET, SPECT and OI, has become an emerging topic in the last ten years. Combinations of imaging modalities have the potential to overcome the respective restrictions of the individual imaging techniques and provide more accurate and complete physiological and anatomical information for diagnosis and therapy. In certain applications, the combination of imaging techniques into a single particle could also offer the benefits of a reduced dose of contrast agent, a shorter procedure time for both patients and scanners, and the assurance that different scans reflect contrast located in the same physiological conditions and spatial position.

Compared with small molecules or bioconjugates widely used in research and the clinic, nanoparticles (NPs) allow an enhanced imaging signal due to their high payload, as well as a high avidity via multiple targeting ligands attached to their surface. In addition, T₂ or T₂* contrast in MRI inherently requires that the contrast agents are particulate. As fluorescent probes, NPs can not only provide more intense and stable emissions (with peaks tuneable from the visible to the near-infrared region), but are generally more thermally stable under laser illumination than organic molecular dyes. The majority of multimodality contrast agents are currently based on superparamagnetic iron oxide NPs whilst a few examples of Gd or Mn containing NPs have also been reported. Since Weissleder et al. reported their pioneering work on multimodal imaging, combinations of NPs and functional polymers or polydentate ligands have been widely applied to obtain multifunctional agents. An alternative approach is hybrid inorganic nanocomposites containing two materials with different properties, such as Fe₃O₄@NaGdF₄ NPs. Although NaYF₄@FeₓO_{y} and Fe₃O₄@LnF₃ were reported as multimodal contrast agents, there was a lack of evidence showing core-shell structures.

Superparamagnetic NPs have been intensively investigated due to their potential applications in biosensors, targeted drug delivery, MRI and hyperthermia.

Unfortunately, these NPs tend to aggregate and form larger secondary particles in order to minimise their surface energy. Moreover, the majority of magnetic NPs are synthesised in organic solvents and coated with organic layer of oleylamine or oleic acid which render them soluble only in non-polar solvents. On the other hand, medical or bio-applications require colloidal stability and dispersability in water and tissue culture environments. Many methods have been developed to obtain a stable colloid of magnetic NPs. Amongst them, coating with polyethyleneglycol (PEG) or Dextran has been widely used, as they are not only hydrophilic and biocompatible but also provide a steric barrier against aggregation, making them hardly recognised by the macrophage-monocytic system. To avoid desorption of the polymeric coating by heating or dilution, one or more functional groups are necessary to bind with the NPs. Such polymers, however, involve a complicated multi-step synthesis approach. Therefore the use of an inorganic shell material that introduces the multimodal functions is desirable and circumvents the need for a designed surface ligand.

There is, therefore, a need in the art for NPs with a well-defined core-shell structure designed for applications in molecular imaging.

According to a first aspect, there is provided a nanoparticle imaging agent (NPIA) comprising an inner magnetic core, and an outer shell disposed substantially around the core, wherein the shell is configured to be radiolabelled.

The inventors have found that it is surprisingly possible to produce the multi-modal nanoparticle imaging agent (NPIA) of the first aspect comprising a magnetic component, which is visible under MRI, and a radiolabel, which is visible under PET. The imaging agent has a well-defined structure, to ensure that the properties do not vary between agents and that the signals of different modalities do not emanate from different agent types with potentially different in vivo locations. The major advantages of these NPIAs as PET/SPECT tracers are the simple and quick radiolabelling process, which is essential for routine clinical use. The in and ex vivo studies in lymph nodes demonstrated the potential advantages of combining imaging modalities using NPs as multi-modal (PET, MRI and optical) imaging agents. In addition, these NPIAs could also potentially act as visual guides during surgery due to their up-conversion fluorescent properties.

As described in the Examples, the NPIA of the first aspect may be produced by a two-step thermolysis process. Preferably, the NPIA is produced by first heating a magnetic metal precursor in a solvent to produce the magnetic core, and then depositing a material layer substantially around the magnetic core to produce the outer shell which can be radiolabelled, to thereby produce the NPIA. The inventors believe that this is an important aspect of the invention.

Hence, in a second aspect, the invention provides a method of preparing a nanoparticle imaging agent (NPIA), the method comprising:—
(i) heating a magnetic metal precursor in a solvent to produce a magnetic core;
(ii) depositing a layer substantially around the magnetic core to produce an outer shell, and
(iii) radiolabelling the shell, to produce a NPIA.

Advantageously, the magnetic core provides the NPIA with the ability to be visible under MRI. The magnetic core may comprise a paramagnetic or superparamagnetic material. For example, the magnetic core may comprise iron, nickel, cobalt or dysprosium or a compound, such as an oxide or alloy, which contains one or more of these elements. In one preferred embodiment, the magnetic core comprises magnetite (Fe₃O₄). In another preferred embodiment, the magnetic core comprises metal-doped iron oxide, for example MFe₂O₄, wherein M is Mn, Fe or Co.

The outer shell of the NPIA preferably comprises a material that can be radiolabelled. Preferably, the outer shell comprises a biocompatible material that has a high affinity for fluoride. For example, the outer shell is ¹⁸F-fluoride absorbent. Preferably, the outer shell comprises NaYF₄ or Al(OH)₃.

Advantageously, use of Al(OH)₃ displays excellent colloidal stability in water. Another benefit of an Al(OH)₃ coating is the high affinity to fluoride anions, as Al³⁺ cations have the strongest interaction with F⁻ anions of all metal cations. The high affinity of NPs to fluoride offers a high labelling efficiency achieved by simply incubating NPs with [¹⁸F]-fluoride solution for 5 minutes, yielding materials which have potential applications as dual-modality contrast agents for MRI/PET, radiotherapy, hyperthermia, cell tracking and vaccine adjuvants. This high affinity to ¹⁸F, together combined with the particle's magnetic core offers potential applications in cancer therapy by combined radiotherapy and hyperthermia, which may kill tumours more efficiently.

Advantageously, the radiolabel on or in the outer shell allows the NPIA to be visible using PET or SPECT. The outer shell may be readily radiolabelled with any radioactive nuclide, such as ¹⁸F, ⁶⁴Cu, ⁸²Rb, ^{99m}Tc, ⁶⁸Ga, ⁸⁹Zr, or ¹¹¹In. It will be appreciated that ⁶⁴Cu is preferred as a positron emitter and that ^{99m}Tc is preferred as a gamma emitter. To label the outer shell, the NPIA is preferably incubated in the presence of the radiolabel, or bisphosphonate-derived conjugate of the radiolabel, in an aqueous solution.

The outer shell may be attached to the magnetic core by physical absorption, by covalent bonding and/or by epitaxial growth. The amount of shell attached to the magnetic core is enough so that the outer shell is disposed substantially around the core. Preferably, the outer shell covers at least 60%, 70%, 80%, 90% or 95% of the outer surface of the core. Preferably, the outer shell covers between 60% to 100%, between 70% to 100%, between 80% to 100%, between 90 to 100%, or between 95 to 100% of the core's surface. It is preferred that the magnetic core is continuously covered (i.e. without spaces) with the shell.

Preferably, the NPIA comprises, or is doped with, a rare earth metal. Preferably, the rare earth metal is fluorescent. Suitable materials which may be used for doping include a lanthanide, especially lanthanide cations, such as ytterbium (Yb), erbium (Er), thulium (Tm) or holmium (Ho) cations. Preferably, the NPIA comprises, or is co-doped with ytterbium (Yb) and another rare earth metal, such as erbium (Er), thulium (Tm) or holmium (Ho) cations. Preferably, the outer shell of the nanoparticle comprises, or is doped with, a rare earth metal. The outer shell may be doped with at least one, two, three, or four rare earth metal materials. Advantageously, the rare earth metal/s allows the nanoparticle imaging agent to be visible using optical imaging techniques.

Accordingly, the NPIA of the invention combines the magnetic core and a fluorescent component with rapid, facile and efficient radiolabelling, under sterile, GMP (Good Manufacturing Practice) conditions with minimal manipulation. The imaging agent allows the inventors to tune the fluorescent properties by doping and optimise the magnetic properties by altering the core-shell ratio or the size and composition of the magnetic core.

Nanoparticles with stronger fluorescent emissions may be produced by deposition of a further doped layer to form a second outer shell disposed around the first outer shell disposed around the inner magnetic core, or by insertion of another layer of low refractive index material between magnetic core and fluorescent layer. The nanoparticle may comprise 1, 2, 3, 4 or 5 shells.

The average diameter of the NPIA may be at least 2 nm, 5 nm, 10 nm, 20 nm, 30 nm, 40 nm, 50 nm, 100 nm, 500 nm or 900 nm.

In a preferred embodiment, the thickness of the outer shell(s) and the size of the magnetic core may be adjusted to optimise certain imaging properties of the NPIA. The average diameter of the core may be at least 1 nm, 2 nm, 3 nm, 4 nm, 5 nm, 6 nm, 7 nm, 8 nm, 9 nm or 10 nm, preferably 4 to 7 nm. The average thickness of the shell may be at least 1 nm, 2 nm, 3 nm, 4 nm, 5 nm, 6 nm, 7 nm, 8 nm, 9 nm or 10 nm, preferably 2 to 4 nm.

The outer shell may comprise one or more ligands. Advantageously, the provision of the ligand serves to stabilise the nanoparticle in vivo, prolong its circulation time, avoid immediate reticulendothelial clearance or facilitates delivery to a target site in vivo. For example, the ligand may target NPs to a tumour cell or a marker expressed by the cells of a certain tissue or organ, e.g. the heart, lungs or kidney. Preferably, the ligands are arranged in a spaced-apart array covering the outer surface of the outer shell. The shell may be functionalised with one species (i.e. the same type) of ligand. However, the shell may be functionalised with two or more species (i.e. different type) of ligand.

The ligand is attached to the outer shell by strong coordinative interactions between phosphate groups of bisphosphonate (BP) and metallic sites on the particle surface. Advantageously, the bisphosphonate affinity of the shell affords the capability for surface derivatisation with targeting molecules or polymers to control solubility and in vivo behaviour or to attach radionuclides for radionuclide imaging.

The ligand may comprise a polymer, which may comprise a polypeptide, a charged protein, a polysaccharide or a nucleic acid. Suitable polymers may comprise any biocompatible natural or synthetic polymer including, but not limited to, chitosan, collagen, gelatine, hyaluronic acid, poly(ethylene glycol) (PEG), bisphosphonate poly(ethylene glycol) (BP-PEG), poly(lactic acid), poly(glycolic acid), poly(epsilon-caprolactone), or poly(acrylic acid). Preferably, the ligand comprises BP-PEG. The outer shell may, therefore, comprise a stabilising ligand, a targeting ligand and a radiolabelling ligand.

In preferred embodiments, the NPIA comprises: (i) an inner magnetic core comprising Fe₃O₄; and (ii) an outer shell comprising radiolabelled NaYF₄. In other preferred embodiments, the NPIA comprises: (i) an inner magnetic core comprising Fe₃O₄; and (ii) an outer shell comprising radiolabelled Al(OH)₃. Preferably, the radiolabel is ¹⁸F.

In one preferred embodiment, the NPIA comprises: (i) an inner magnetic core comprising cobalt-doped Fe₃O₄; and (ii) an outer shell comprising radiolabelled NaYF₄, and doped with ytterbium (Yb) and erbium (Er).

In another preferred embodiment, the nanoparticle imaging agent comprises: (i) an inner magnetic core comprising Fe₃O₄; and (ii) an outer shell comprising radiolabelled NaYF₄, and doped with ytterbium (Yb) and thulium (Tm).

In yet another preferred embodiment, the nanoparticle imaging agent comprises: (i) an inner magnetic core comprising Fe₃O₄; and (ii) an outer shell comprising radiolabelled NaYF₄, and doped with ytterbium (Yb) and holmium (Ho).

The key properties of these NPIAs are that they are magnetic, fluorescent, and have high affinity to a radiolabel, such as ¹⁸F, and a radioactive metal bisphophonate conjugate. Advantageously, the NPIAs are multi-functional, readily radiolabelled, uniform in size and morphology, and can be synthesised in a single container.

As described in Examples 2 and 4, the inventors have demonstrated that the NPIAs of the invention can be effectively used in PET, MRI and fluorescence imaging techniques.

Thus, in a third aspect, there is provided use of the nanoparticle imaging agent (NPIA) of the first aspect, in an imaging technique.

The imaging technique may be selected from: PET, SPECT, MRI or fluorescence imaging.

According to a fourth aspect, there is provided the nanoparticle imaging agent (NPIA) according to the first aspect, for use in diagnosis.

According to a fifth aspect, there is provided the nanoparticle imaging agent (NPIA) according to the first aspect, for use in surgery.

It will be appreciated that the NPIA of the invention can be used as a biosensor in a range of different biological imaging applications. For example, the nanoparticle is preferably used in PET, SPECT, MRI or fluorescence imaging techniques, as a biolabel. In particular, the NPIA can be used for cell labelling, cell tracking, macrophage imaging and atherosclerosis imaging.

Thus, in a sixth aspect, there is provided use of the nanoparticle imaging agent (NPIA) of the first aspect, as a biolabel.

In a seventh aspect, there is provided a biolabel comprising the nanoparticle imaging agent (NPIA) according to the first aspect.

In Example 2, the inventors have shown that after IV injection, the NPIAs can be used to analyse liver function. In addition, as also discussed in Example 2, the inventors have also demonstrated that the NPIAs of the invention can be effectively used in the accurate location and identification of lymph nodes and detection of the pathology within them before surgery, during surgery, and subsequently during pathological examination of excised nodes.

Thus, in a eighth aspect, there is provided the nanoparticle imaging agent (NPIA) according to the first aspect, for use in therapy, and preferably as a medicament.

Examples of diseases that may be treated include inflammatory disease, such as atherosclerosis or arthritis, solid tumors, haematological diseases and malignancies and autoimmune diseases.

The inventors believe that the NPIA will be useful as a vaccine adjuvant, especially in embodiments where the outer shell is Al(OH)₃.

According to a ninth aspect there is provided use of the nanoparticle imaging agent (NPIA) according to the first aspect, as an adjuvant for a vaccine. Preferably, the shell comprises Al(OH)₃.

All features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:—

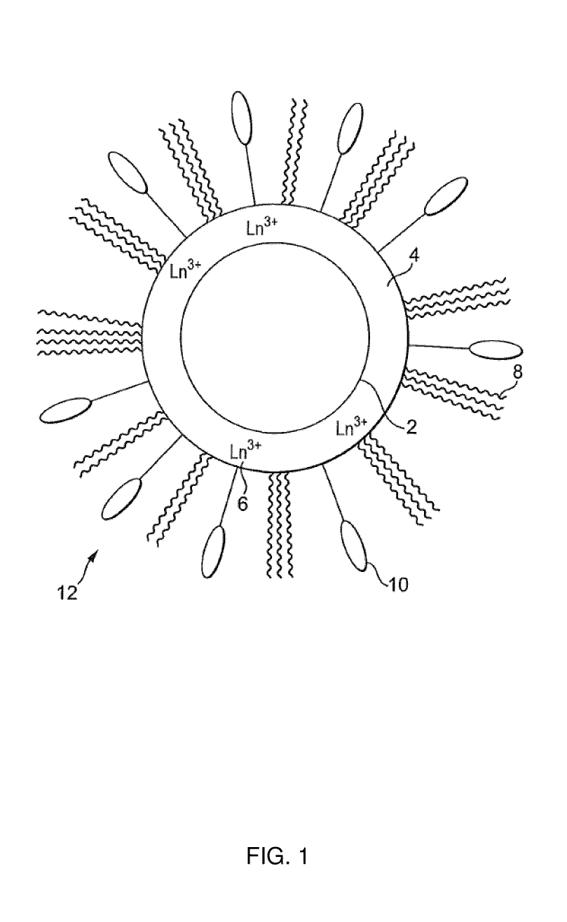

FIG. 1 illustrates a schematic representation of one embodiment of a nanoparticle;

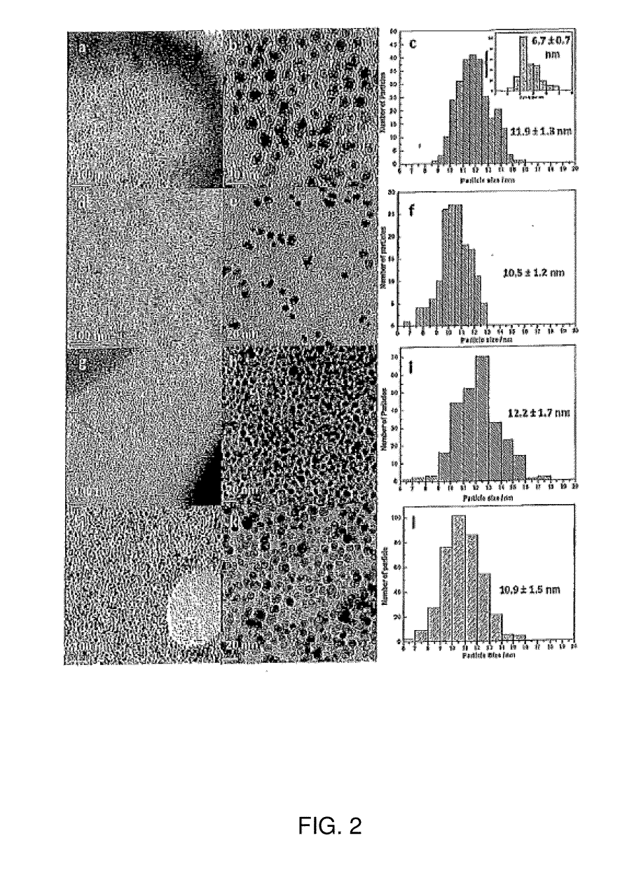

FIG. 2 shows TEM images and the size distribution of CoₓFe₃₋ₓO₄@NaYF₄(Yb, Er) NPIAs obtained (a to c) at 270° C. and their magnetic cores (inset); (d to f) at 300° C.; (g to i) at 340° C.; and (j to l) TEM images and size distribution of Fe₃O₄@NaYF₄(Yb, Tm) obtained at 340° C.;

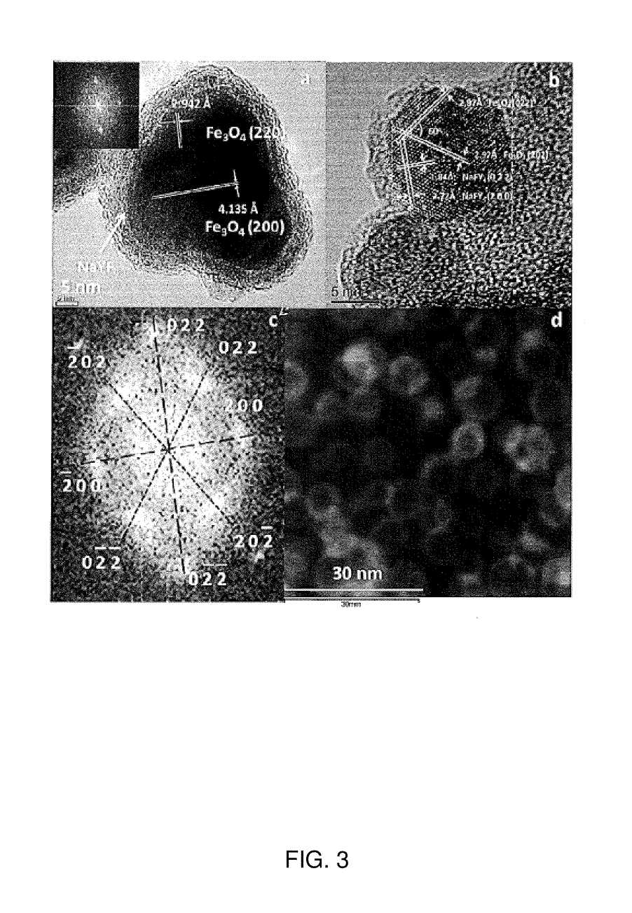

FIG. 3 shows HRTEM micrographs of NPIAs: (a) HTRM image revealed the core-shell structure of NPIA CoₓFe₃₋ₓO₄@NaYF₄(Yb, Er), atomic lattice fringes 2.942 Å and 4.135 Å corresponded to (220) and (002) planes of Fe₃O₄ respectively, the inset is a fast Fourier transform of the micrograph; (b) HRTEM images of Fe₃O₄@NaYF₄(Yb, Tm); (c) fast Fourier transform of the selected area in FIG. 3b showed two sets of diffraction patterns; and (d) High Angle Annual Dark Field image of Fe₃O₄@NaYF₄(Yb, Tm), showing the Z contrast difference between the shell and core of particles induced by a slightly higher average atomic number in the shell after doping with heavy atoms Yb and Tm;

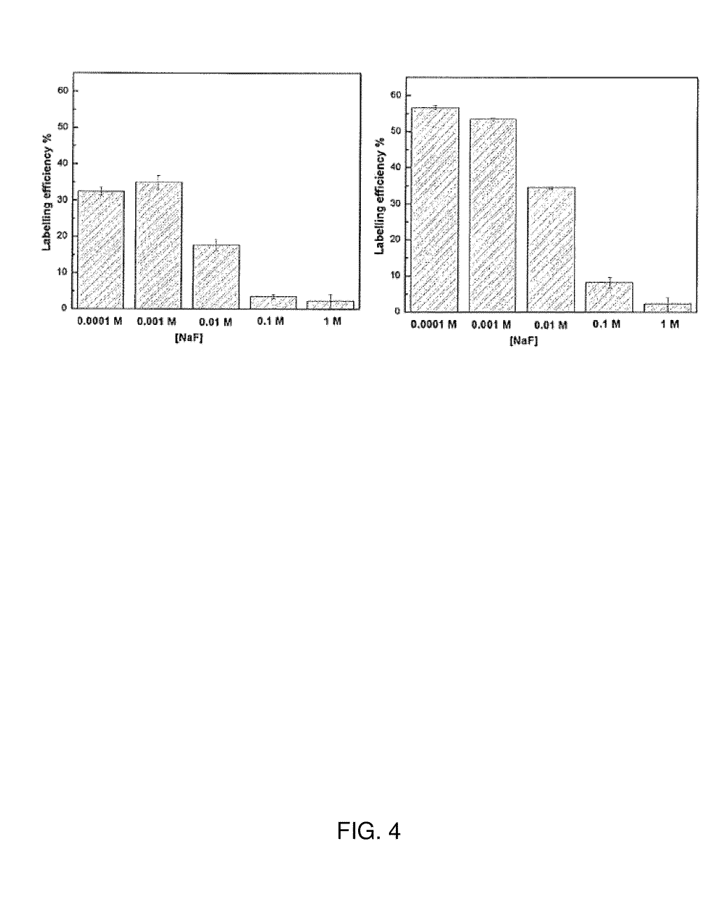

FIG. 4 shows graphs depicting ¹⁸F labelling of 0.1 mg MSA functionalised CoₓFe₃₋ₓO₄@NaYF₄(Yb 20%, Er 2%) in the presence of NaF; left, CoₓFe₃₋ₓO₄@NaYF₄(Yb 20%, Er 2%) NPIAs obtained at 300° C.; right, CoₓFe₃₋ₓO₄@NaYF₄(Yb, Er) NPIAs obtained at 340° C.;

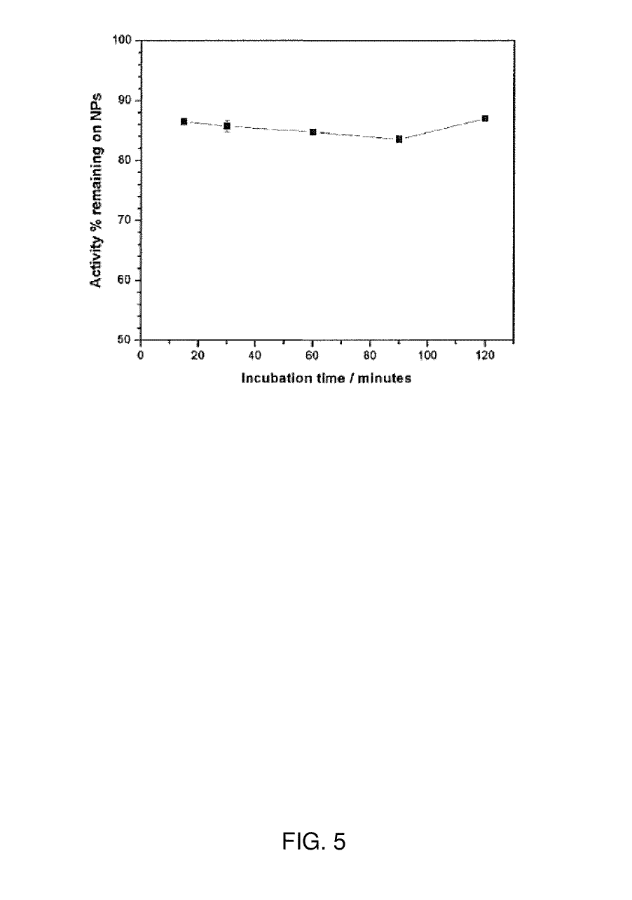

FIG. 5 shows a graph depicting stability of ¹⁸F radiolabelled CoₓFe₃₋ₓO₄@NaYF₄(Yb, Er)-BP-PEG in serum;

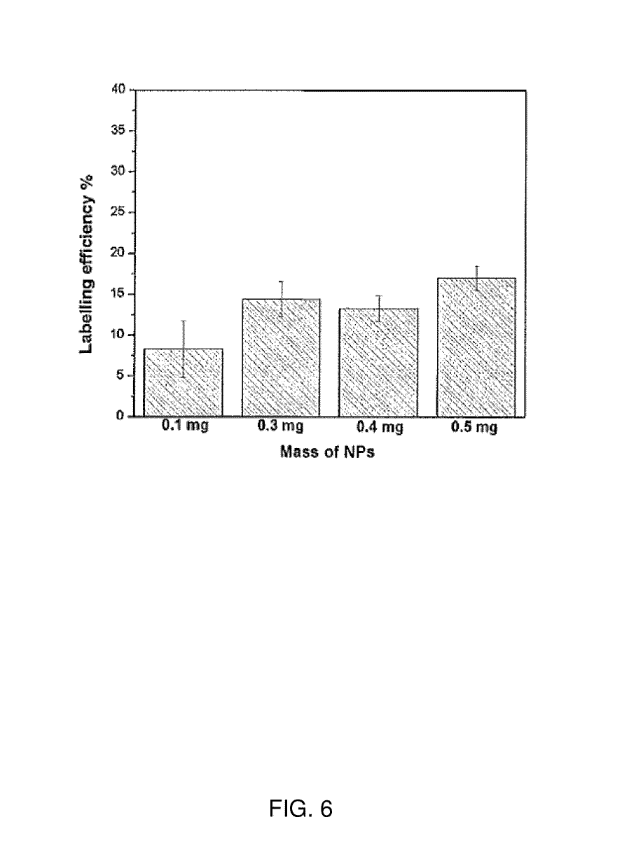

FIG. 6 shows a graph depicting ^{99m}Tc-MDP labelling of CoₓFe₃₋ₓO₄@NaYF₄(Yb, Er)-MSA;

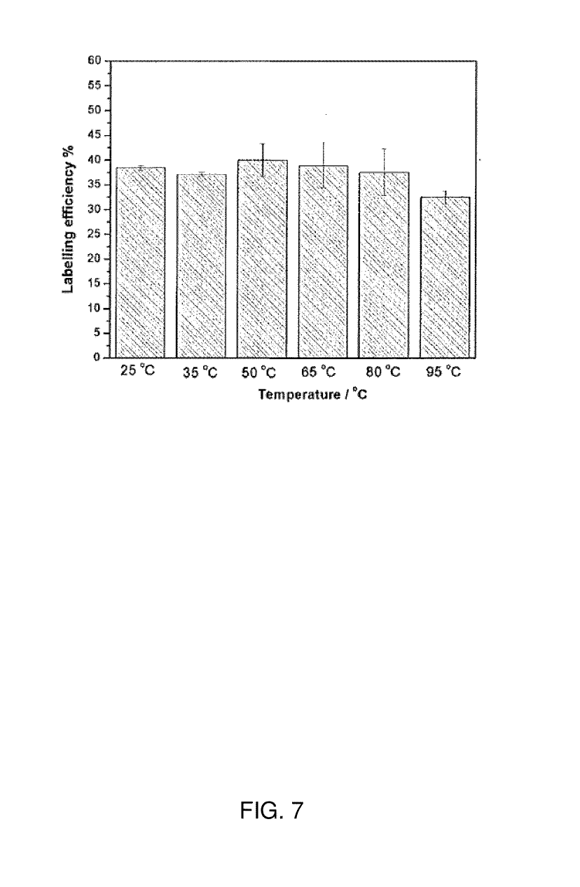

FIG. 7 shows a graph depicting ¹⁸F Labelling efficiency of CoₓFe₃₋ₓO₄@NaYF₄(Yb, Er)-BP-PEG obtained by incubating the solution containing 0.1 mg CoₓFe₃₋ₓO₄@NaYF₄(Yb, Er)-BP-PEG at different temperatures (from 25 to 95° C.) for 5 minutes;

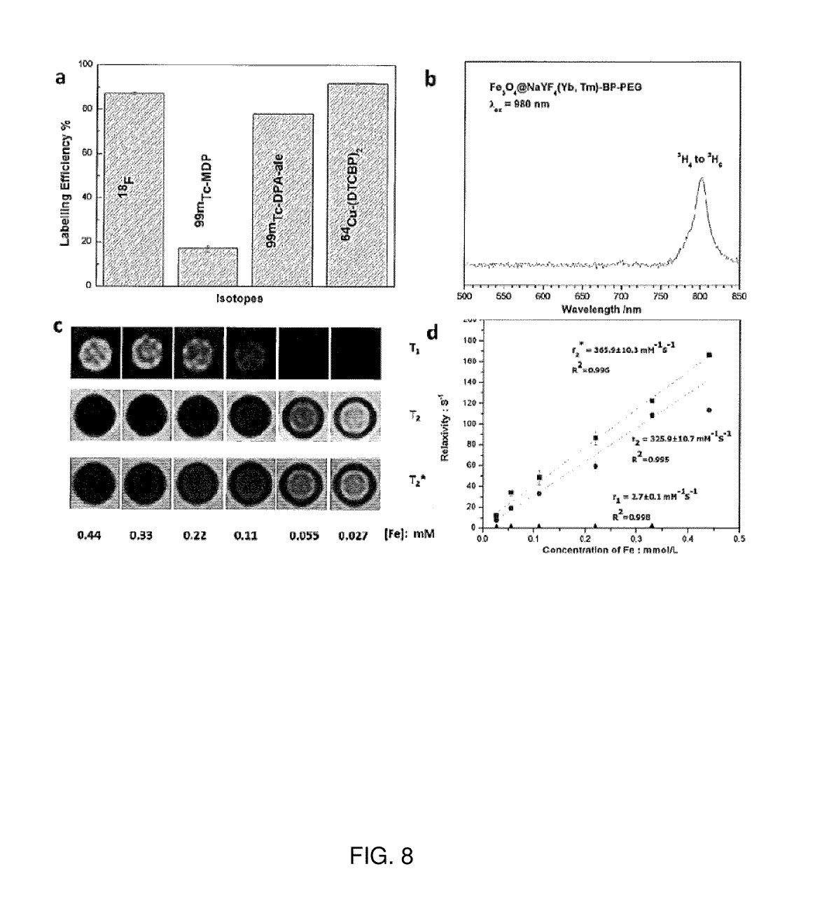

FIG. 8(a) shows a graph depicting the radiolabelling efficiency of MSA-functionalised CoₓFe₃₋ₓO₄@NaYF₄(Yb, Er)-MSA NPIAs (1 mg) with [¹⁸F]-fluoride and radiometal-bisphosphonate conjugates at room temperature; (b) up-conversion spectra of sample Fe₃O₄@NaYF₄(Yb, Tm)-BP-PEG under excitation by a 980 nm laser, showing emission at 800 nm; (c) MRI images (T₁, T₂, T₂*) of aqueous solutions containing Fe₃O₄@NaYF₄(Yb, Tm)-BP-PEG at different concentrations; and (d) curve of relaxivity against the concentration of Fe for Fe₃O₄@NaYF₄(Yb, Tm)-BP-PEG at 3 T. Fe concentration was measured by ICP-MS;

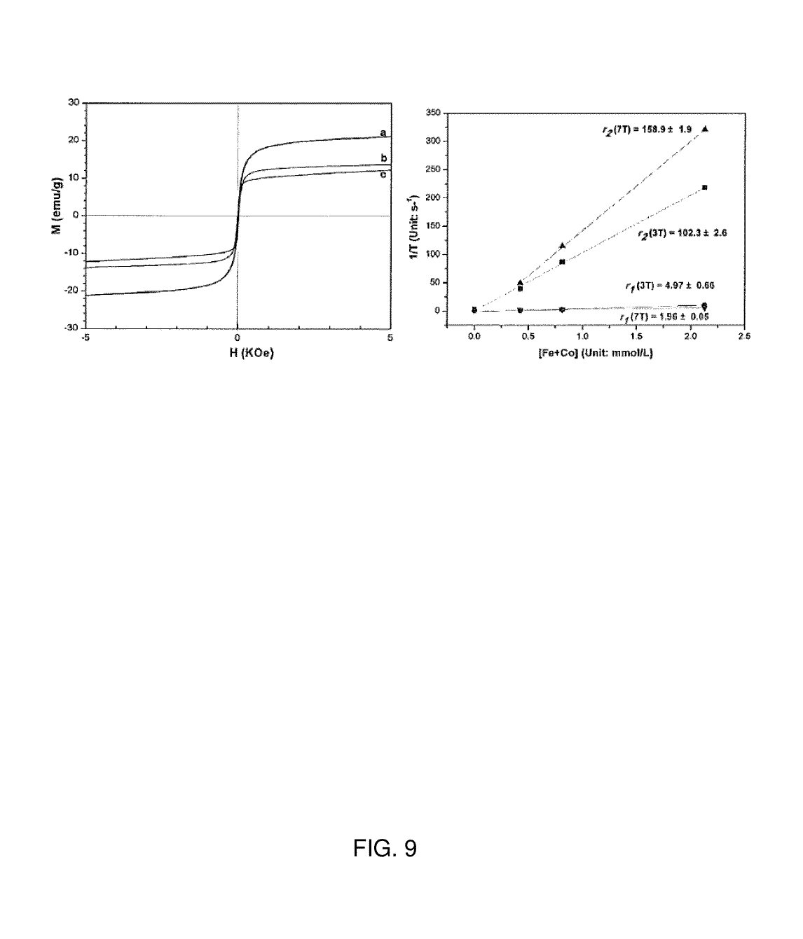

FIG. 9 shows left, graph depicting magnetisation as a function of applied field at room temperature for NPIAs: (a) CoₓFe₃₋ₓO₄@NaYF₄(Yb, Er); (b) CoₓFe₃₋ₓO₄@NaYF₄(Yb, Er)-BP-PEG (10K); and (c) Fe₃O₄@NaYF₄(Yb, Tm). Right, graph depicting T₁⁻¹ and T₂⁻¹ versus concentration [Fe+Co] of aqueous solution of CoₓFe₃₋ₓO₄@NaYF₄(Yb, Er)-BP-PEG obtained under magnetic field 3 T and 7 T respectively at room temperature. Values of saturated magnetic moments were calculated on the basis of the particle mass. The concentration of Fe and Co was measured by ICP-MS;

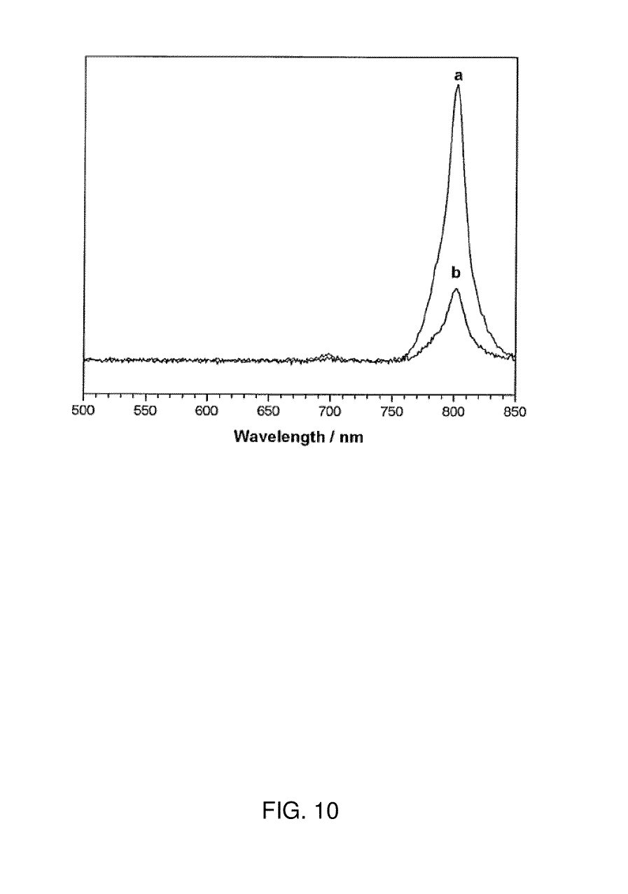

FIG. 10 depicts up-conversion fluorescent spectrum of: (a) Fe₃O₄@NaYF₄(Yb, Tm)@NaYF₄-BP-PEG; and (b) Fe₃O₄@NaYF₄(Yb, Tm)-BP-PEG, showing an improved fluorescence after deposition of another NaYF₄ layer;

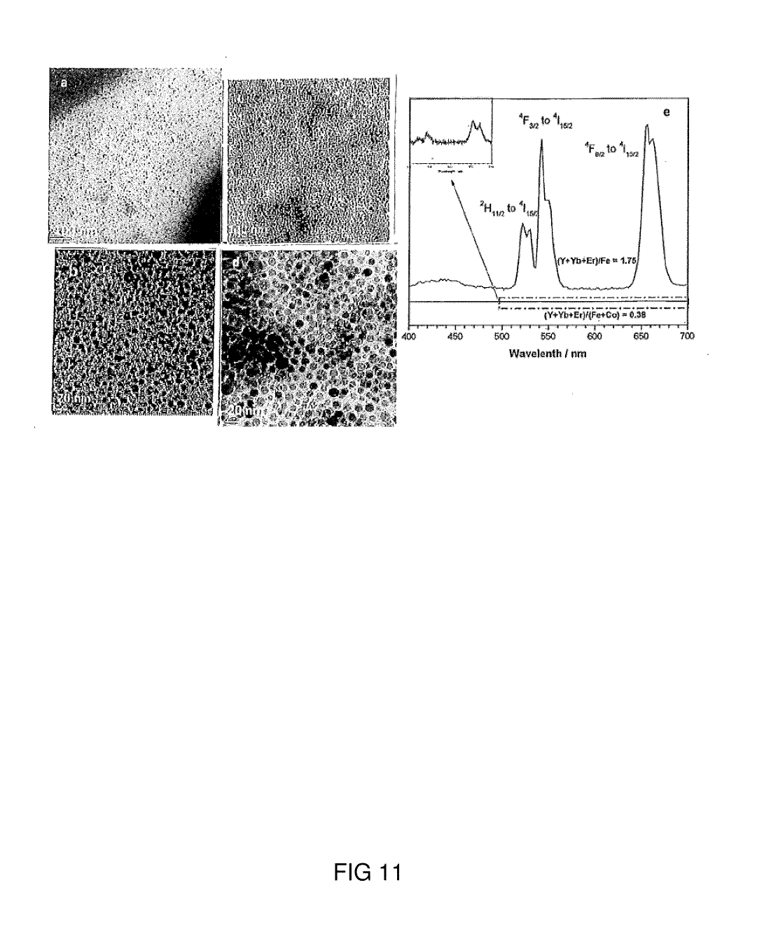

FIG. 11 depicts in (a) and (b) TEM images of CoₓFe₃₋ₓO₄@NaYF₄(Yb, Er)NPIAs, (c) and (d) Fe₃O₄@NaYF₄(Yb, Er) NPIAs; and (e) Up-conversion fluorescent spectrum of Fe₃O₄@NaYF₄(Yb, Er) and CoₓFe₃₋ₓO₄@NaYF₄(Yb, Er) (inset), showing an improved fluorescence after increasing the ratio of rear earth cations to magnetic cation(s) Fe and Co. The compositional study of NPIAs were carried out by ICP-MS;

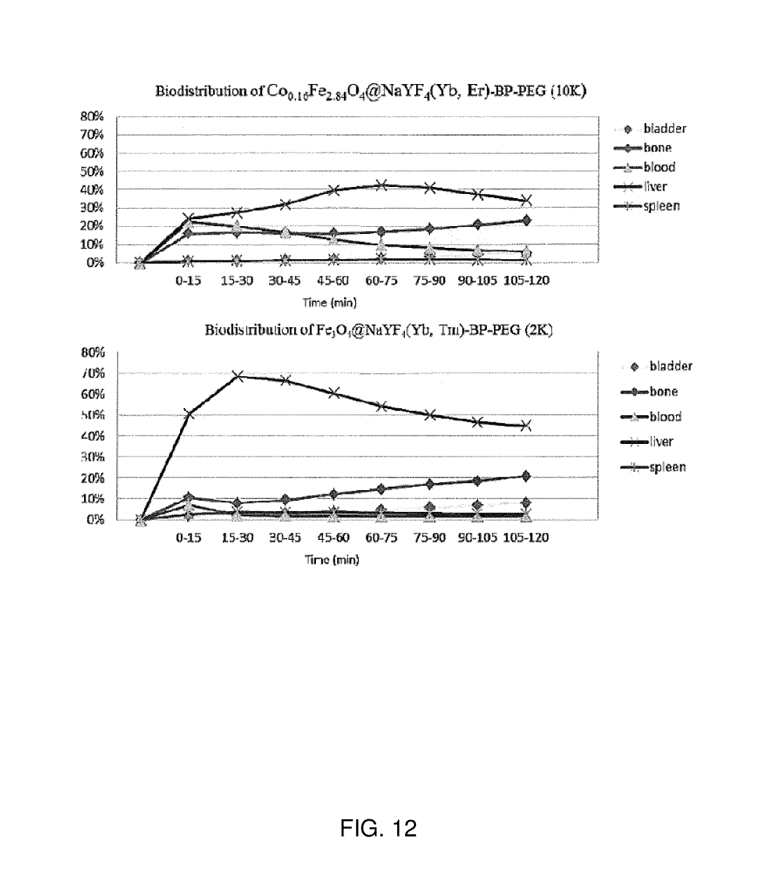

FIG. 12 shows a graph depicting dynamic biodistribution of ¹⁸F-labelled CoₓFe₃₋ₓO₄@NaYF₄(Yb, Er)-BP-PEG (10K) (upper) and ¹⁸F-labelled Fe₃O₄@NaYF₄(Yb, Tm)-BP-PEG (2K) (bottom) quantified by PET. The diagram shows time curves of % of injected radioactivity in specific organs (bladder, bone, blood, liver, spleen);

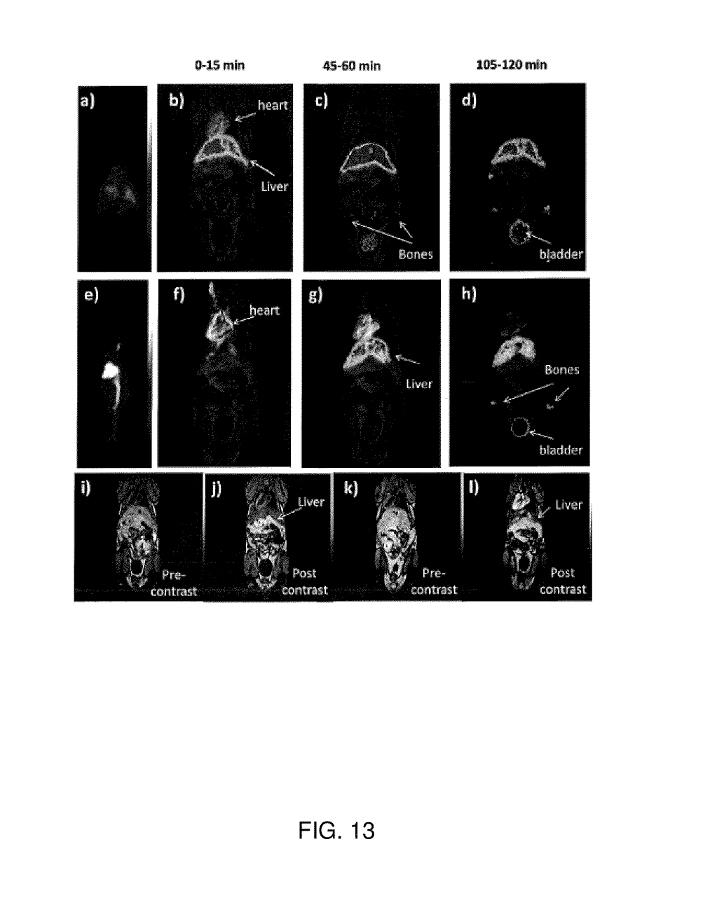

FIG. 13 shows PET/MRI images of the dynamic bio-distribution of ¹⁸F-labelled CoₓFe₃₋ₓO₄@NaYF₄(Yb, Er)-BP-PEG (10K) and ¹⁸F-labelled Fe₃O₄@NaYF₄(Yb, Tm)-BP-PEG (2K). MR images were taken immediately after PET scan, 2 hours post the injection of NPIAs, and they were fused with PET images taken at three different time intervals (0-15 mins, 45-60 mins, and 105-120 mins). (a) Whole body PET image showing up-take of radiolabelled positive charged ¹⁸F-labelled Fe₃O₄@NaYF₄(Yb, Tm)-BP-PEG (2K) (maximum intensity projection, 30-45 mins); (b) PET/MRI fused image at 0-15 mins; (c) PET/MRI fused image at 45-60 mins; (d) PET/MRI fused image at 105-120 mins; (e) whole body PET image showing uptake of radiolabelled negative charged ¹⁸F-labelled CoₓFe₃₋ₓO₄@NaYF₄(Yb, Er)-BP-PEG (10K) (maximum intensity projection, 30-45 mins); (f) PET/MRI fused image at 0-15 mins; (g) PET/MRI fused image at 45-60 mins; and (h) PET/MRI fused image at 105-120 mins; (i) MR image of the mouse prior to the injection of ¹⁸F-labelled Fe₃O₄@NaYF₄(Yb, Tm)-BP-PEG (2K) NPIAs; (j) MR image of the mouse post the injection of ¹⁸F-labelled Fe₃O₄@NaYF₄(Yb, Tm)-BP-PEG (2K) NPIAs; (k) MR image of the mouse prior to the injection of ¹⁸F-labelled CoₓFe₃₋ₓO₄@NaYF₄(Yb, Er)-BP-PEG (10K) NPIAs; (l) MR image of the mouse post the injection of ¹⁸F-labelled CoₓFe₃₋ₓO₄@NaYF₄(Yb, Er)-BP-PEG (10K) NPIAs. The middle series of images (e) to (h) shows predominantly blood pool retention of the labelled NPIAs giving way to liver uptake and then release of radioactivity which appears in bone and bladder;

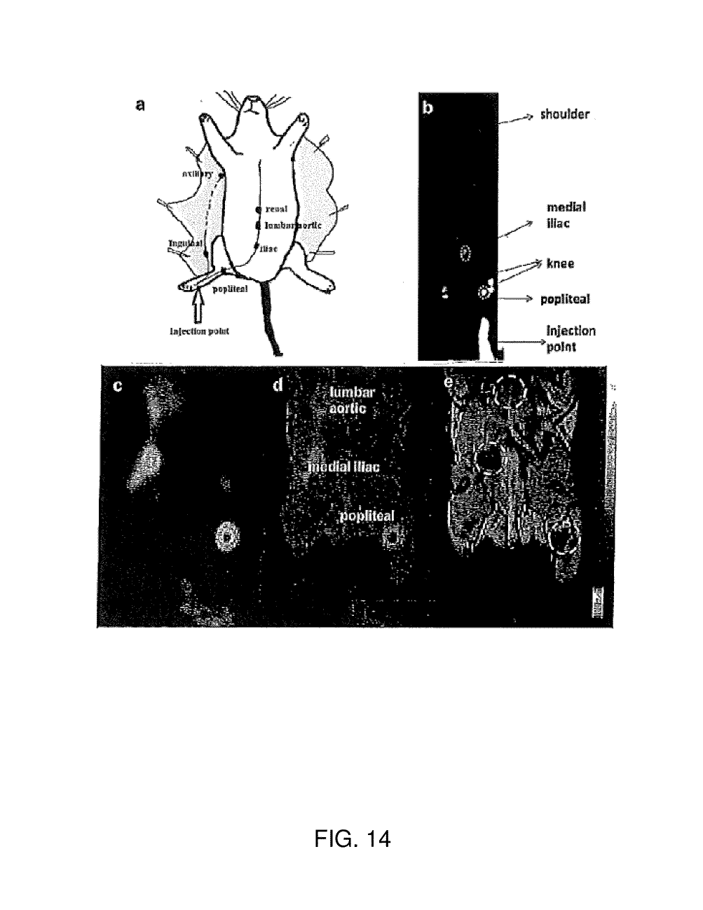

FIG. 14 shows PET/MRI images of a normal young C57BL/6 mouse showing lymph nodes (LNs) with dual contrast provided by ¹⁸F-labelled CoₓFe₃₋ₓO₄@NaYF₄(Yb, Er)-BP-PEG: (a) schematic diagram showing the connections between lymph nodes and the injection point (mouse in supine position); (b) whole body PET image showing uptake of radiolabelled NPIAs 7 hours post injection (maximum intensity projection, mice in prone position); (c) PET image showing popliteal, iliac and renal LNs (coronal section); (d) PET/MRI fused image (coronal section); and (e) MRI image with darkening contrast at popliteal and iliac LNs (coronal section). Some bone uptake of radioactivity is observed in (b), (c) and (d) due to gradual release of fluoride from the particles;

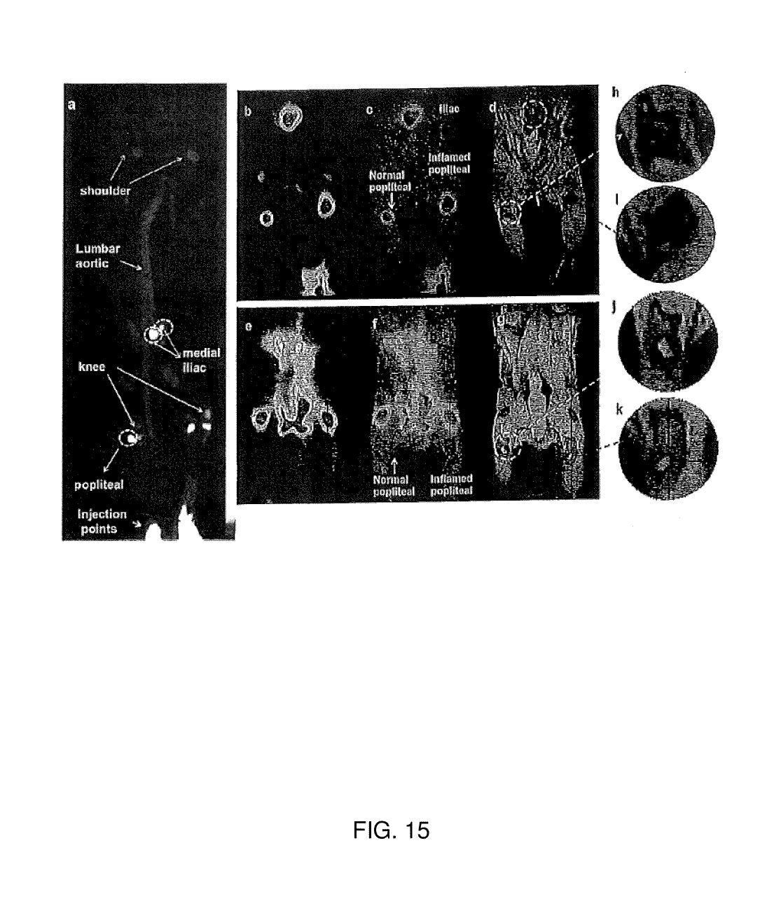

FIG. 15 shows LN PET/MRI imaging of a mouse with inflamed right leg using ¹⁸F-labelled Fe₃O₄@NaYF₄(Yb, Tm)-BP-PEG NPIAs (a-d) or with [¹⁸F]-fluoride only (e-g): (a) whole body PET image showing uptake of radiolabelled NPIAs (maximum intensity projection) after injection via footpad; bone uptake was observed due to gradual release of fluoride from NPIAs; (b) PET image showing popliteal and iliac lymph nodes (coronal section); (c) PET/MRI fused image (coronal section); (d) MR image (coronal section) with darkening contrast inside popliteal lymph node at left-rear (white circle) and ‘outside’ lymph node at the inflamed right-rear (circle) induced by injection of 30 μL 0.67 mg/mL lipopolysaccharide (LPS) 18 hours prior to imaging, and at iliac lymph node; (e) PET image following injection of [¹⁸F]-fluoride via footpad showing no contrast in lymph nodes in the absence of NPIAs and prominent uptake by skeleton; (f) PET/MRI fused image following injection of [¹⁸F]-fluoride, showing no radioactivity associated with lymph nodes; (g) MR image showing no difference between normal popliteal lymph node at left-rear leg (white circle) and the inflamed lymph node at right-rear leg induced by injection of 30 μL 0.67 mg/mL LPS 18 hours prior to imaging; and (h-k) enlarged MR images of corresponding lymph nodes;

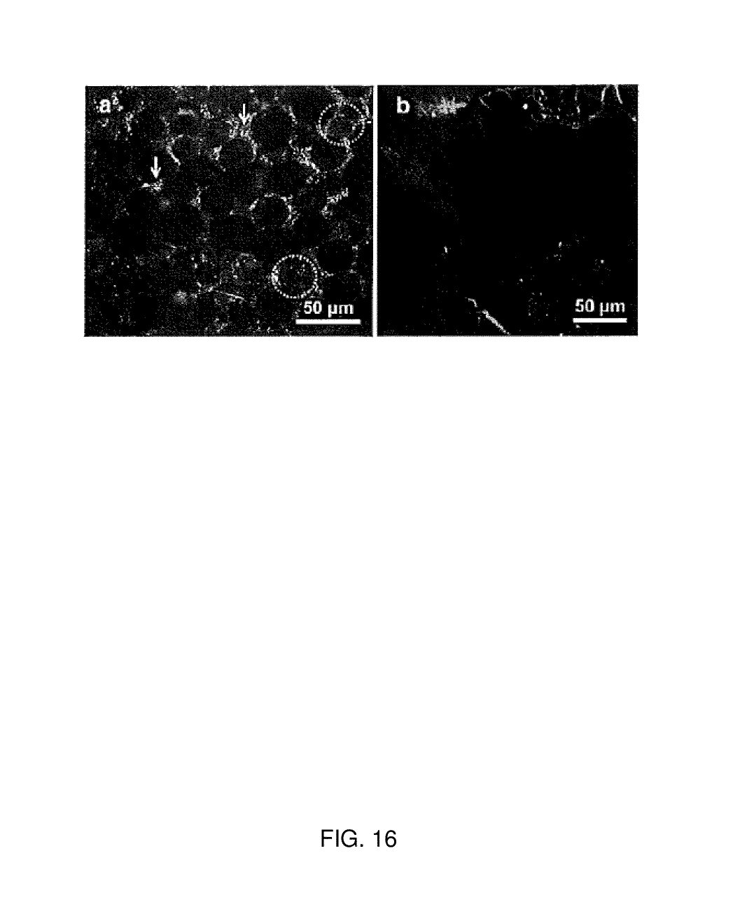

FIG. 16 shows Ex vivo fluorescent imaging of lymph nodes under a laser of 980 nm: (a) inflamed popliteal lymph node excised from the mouse with injection of [¹⁸F]-fluoride radiolabelled Fe₃O₄@NaYF₄(Yb, Tm)-BP-PEG NPIAs, showing a strong fluorescence from NPIAs between the lipid droplets of fatty cells (indicated by arrows), in the cytoplasm (inside the circles) and some noise (red blots), in deep step of 30 μm; and (b) inflamed popliteal lymph node excised from the control mouse with injection of [¹⁸F]-fluoride only, showing faint granular fluorescence from actin filaments and greenish fluorescent collagen fibres;

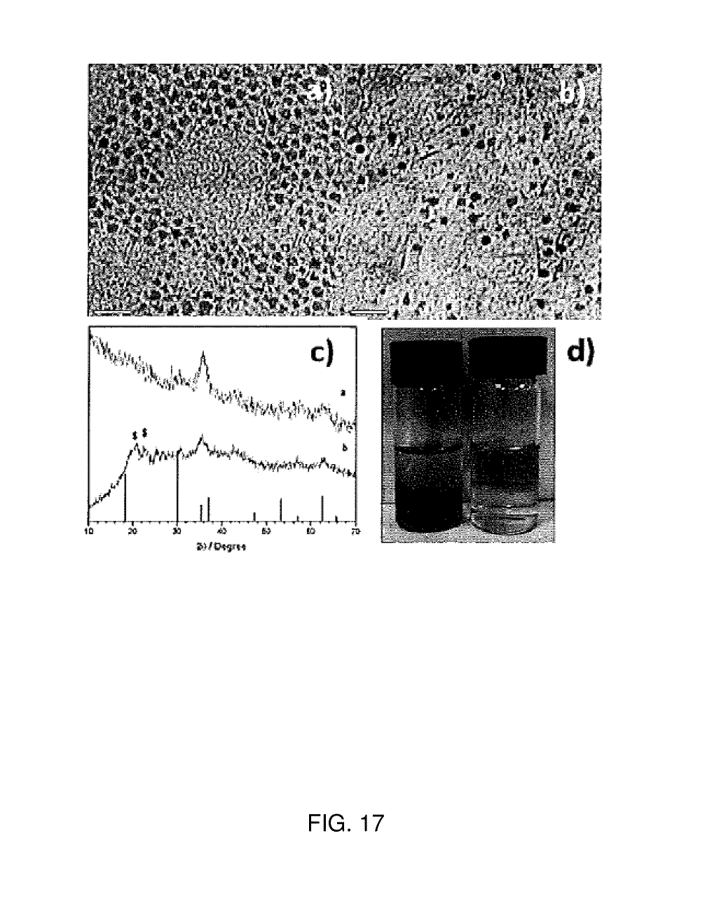

FIG. 17 shows (a) TEM images of MnFe₂O₄ NPIAs isolated from hexane; (b) TEM image of MnFe₂O₄@Al(OH)₃ NPIAs isolated from water; (c) XRD patterns of MnFe₂O₄ and MnFe₂O₄@Al(OH)₃ NPIAs. The black lines show the reference XRD pattern calculated from the published crystallographic data of Fe₃O₄; (d) digital photographs of MnFe₂O₄ (right) and MnFe₂O₄@Al(OH)₃ (left) NPIAs in the mixture of hexane (upper layer) and water (bottom layer), showing that MnFe₂O₄ is soluble only in hexane whereas MnFe₂O₄@Al(OH)₃ is soluble only in water. Scale bar in 20 nm. $ represents the peak of Al(OH)₃ (nordstrandite phase);

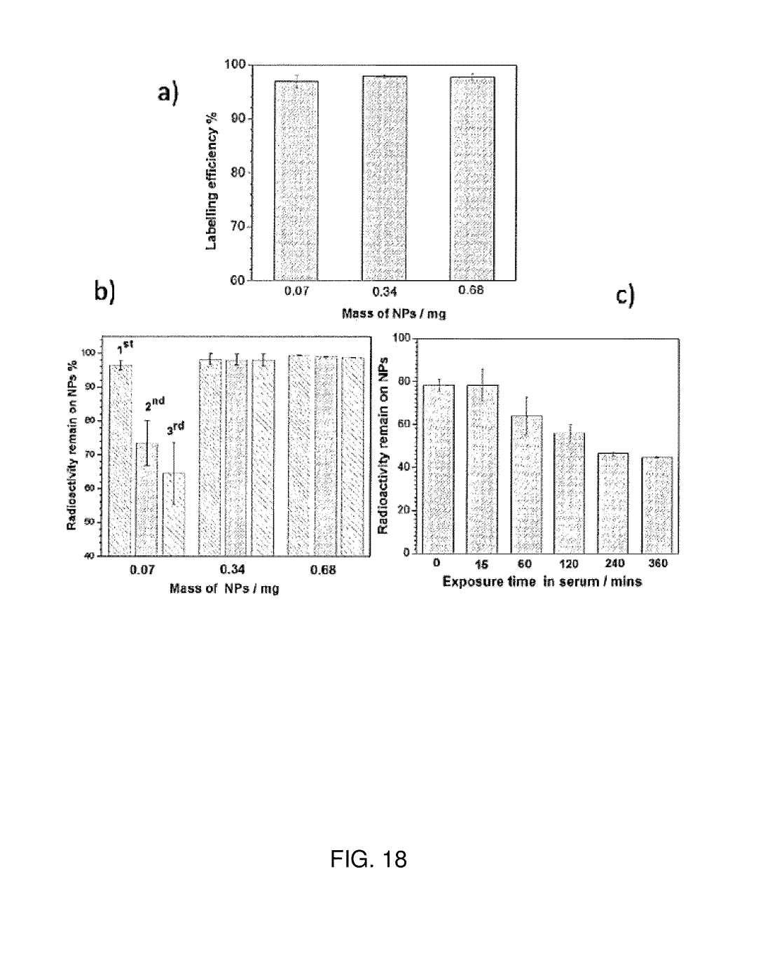

FIG. 18 depicts graphs showing (a) [¹⁸F]-fluoride radiolabelling of MnFe₂O₄@Al(OH)₃ NPIAs in water; (b) the amount of radioactivity remaining on labelled NPIAs after washing with water for 1, 2 or 3 times respectively; and (c) the amount of radioactivity remaining on NPIAs after incubation in human serum for a period of different times (from 0 to 360 mins);

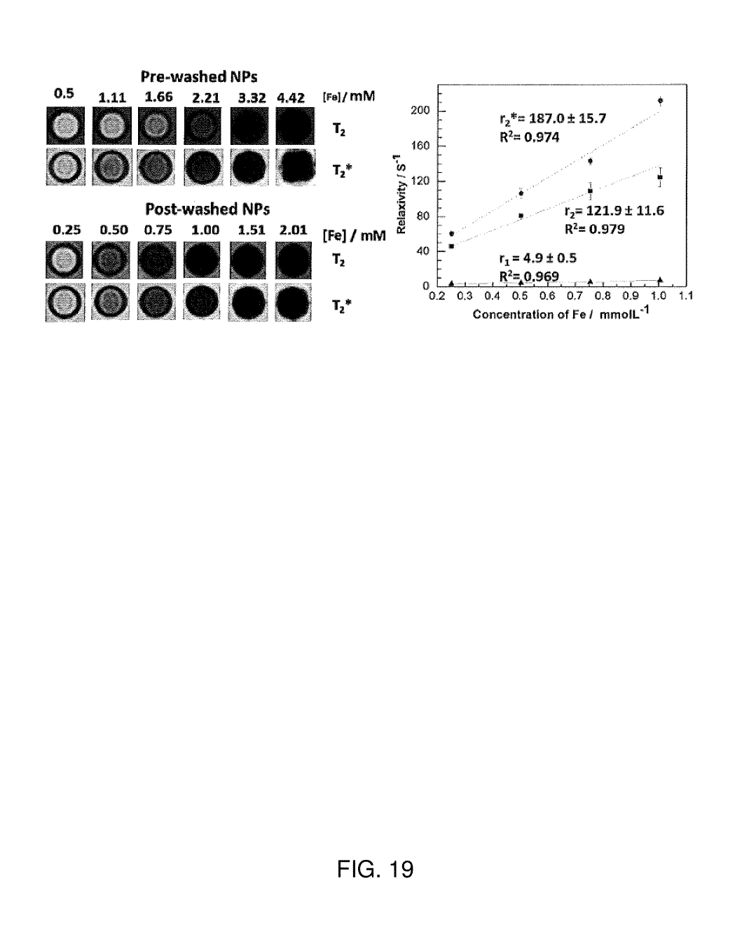

FIG. 19 depicts (left), T₂ and T₂* weighted MR images of aqueous solution containing Fe₃O₄@Al(OH)₃ (1:2) NPIAs; (right), the curves of relaxivity against concentration at 3 T. The concentration of Fe was measured by ICP-MS; and

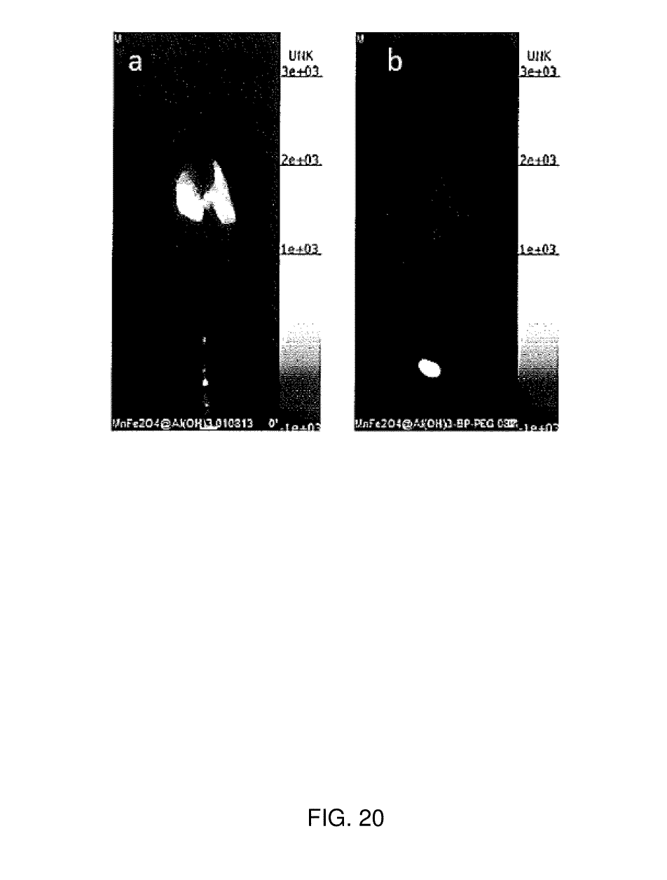

FIG. 20 depicts (a) in vivo PET images of a normal young C57BL/6 mouse using ¹⁸F-labelled MnFe₂O₄@Al(OH)₃; and (b) MnFe₂O₄@Al(OH)₃-BP-PEG. Whole body PET image shows up-take of radiolabeled NPs 15 minutes post injection (maximum intensity projection, mice in prone position). Non-PEGylated nanoparticles (left) are prone to aggregation and thus accumulate in lung; PEGylated nanoparticles (right) are protected from aggregation and escape trapping in lung and activity is seen in blood pool, liver and spleen and skeleton.

### EXAMPLES

The invention will now be described by way of illustration only in the following examples.

The inventors have developed novel NPIAs of uniform size and morphology, with a well defined core/shell structure, having a magnetic core for use in MRI, and a shell that can be readily radiolabelled for use in PET/SPECT and is also rare earth doped for use in fluorescent imaging.

Referring to FIG. 1, there is shown a schematic illustration of one embodiment of a NPIA (12) for targeted multimodality molecular imaging. The NPIA (12) comprises an inner magnetic core (2), an outer shell (4) which can be radiolabelled, and is doped with rare earth elements (6). Also shown are stabilising ligands (8) and targeting ligands (10).

### Example 1

### Synthesis, Structure and Morphology of Nanoparticles

### Synthesis

Typically, oleylamine-coated CoₓFe₃₋ₓO₄@NaYF₄(Yb 20%, Er 2%) NPIAs were first synthesised via a two-step thermolysis. Metal precursors Fe(CO)₅ and Co(acac)₂ (or Co₂(CO)₉) were heated at 250° C. in a solvent mixture of 1-octadecene and oleylamine under N₂ for 1 hour to form CoₓFe₃₋ₓO₄NPs, and a co-doped NaYF₄ layer was deposited during a subsequent decomposition of lanthanide and sodium trifluoroacetate salt at different temperatures up to 340° C. Fe₃O₄@NaYF₄(Yb 20%, Tm 5%) NPIAs were synthesised by a similar procedure, using Fe(CO)₅ and corresponding trifluoroacetate salts as precursors. As shown in FIG. 2, transmission electron microscope (TEM) images revealed that the NPIAs obtained under different temperature conditions shared a similar size and morphology.

CoₓFe₃₋ₓO₄@NaYF₄(Yb, Er) NPIAs, 11.9±1.3 nm diameter were obtained at 270° C., 10.5±1.3 nm at 300° C., 12.2±1.7 nm at 340° C. and 10.9±1.5 nm for Fe₃O₄@NaYF₄(Yb, Tm) at 340° C. The core-shell structure of CoₓFe₃₋ₓO₄@NaYF₄(Yb, Er) NPs obtained at 270° C. and 300° C. can be seen clearly even at low magnification in TEM images, as shown in FIGS. 2b and 2e. The average size of the core was measured using TEM as 6.7±0.7 nm for CoₓFe₃₋ₓO₄@NaYF₄(Yb, Er) NPIAs obtained at 270° C., as shown in FIG. 2c (inset), which is comparable with the size of iron oxide NPIAs obtained previously under similar conditions.

### Structure and Morphology

X-ray powder diffraction (XRD) patterns implied that the NPIAs consisted of two phases, Fe₃O₄ (or CoFe₂O₄) and α-NaYF₄. A small amount of β-NaYF₄ was found in the samples obtained at 340° C., which is not unexpected as β-NaYF₄ is favoured over α-NaYF₄ at high temperature. The high resolution transmission electron micrograph (HRTEM) in FIG. 3a confirmed the core-shell structure of CoₓFe₃₋ₓO₄@NaYF₄(Yb, Er), and the electron diffraction pattern indicated the crystalline nature of the CoₓFe₃₋ₓO₄ core. The atomic lattice fringes of 2.942 Å and 4.135 Å were associated with (220) and (002) planes, respectively, of the cubic Fe₃O₄ phase. The doping of Co into the Fe₃O₄ lattice, and of Yb and Er into NaYF₄ lattice, was confirmed by energy dispersive X-ray spectroscopy (EDX). Despite the presence of heavy atoms Yb and Er, the shell appeared brighter on bright field TEM images, since the contrast is determined by the thickness and crystallinity of the specimen, as well as its elemental composition. HRTEM studies of NPIAs Fe₃O₄@NaYF₄(Yb, Tm) showed atomic lattice fringes of 2.97 Å associated with the (022) and (202) planes of cubic Fe₃O₄ and 1.72 Å and 2.94 Å corresponding to the (022) and (200) planes of cubic NaYF₄ respectively, as shown in FIG. 3b. The angle between the (022) and (202) planes was calculated as 60°, which is consistent with the value measured on HRTEM images. The electron diffraction patterns were obtained by the fast Fourier transform analysis of the HRTEM images. Two sets of diffraction patterns for (Fe₃O₄ and NaYF₄—) were obtained, and each spot was assigned as indicated in FIG. 3c. Analysis of the electron diffraction patterns indicated that core-shell structures were formed by growing the (01-1) plane of NaYF₄ on the (11-1) plane of Fe₃O₄ with a rotation angle of 30°. High angle annular dark field (HADDF, or Z contrast) imaging was employed to investigate the structure of NP Fe₃O₄@NaYF₄(Yb, Tm), as its contrast was strongly dependant on average atomic number of the specimen but insensitive to its thickness. The HAADF image of Fe₃O₄@NaYF₄(Yb, Tm) NPIAs in FIG. 3d clearly showed a core/shell structure. Yb- and Tm-co-doped NaYF₄ shells appeared brighter than the Fe₃O₄ cores.

In addition to EDX, compositional studies were also carried out by X-ray photoelectron spectroscopy (XPS) and inductively coupled plasma mass spectrometry (ICP-MS). While EDX results implied that the cores contained mainly Fe, the global sample is rich in Yb and Y. By comparing the relative content of Fe, Co, Y, Yb and Er obtained by ICP-MS and XPS, it was clear that dramatically less Fe and Co was detected by the surface technique (XPS), than by ICP-MS or EDX. This is consistent with the proposed core-shell structure observed on TEM.

The oleylamine-coated CoₓFe₃₋ₓO₄@NaYF₄(Yb, Er) and Fe₃O₄@NaYF₄(Yb, Tm) core-shell NPIAs described above were converted to a water-soluble form by ligand exchange with either bisphosphonate polyethylene glycol conjugates (BP-PEG) or mercaptosuccinic acid. The appearance in the IR spectrum of the new feature due to C═O at 1711 cm⁻¹ and characteristic peaks associated with the PEG chain at 1109, 958 and 837 cm⁻¹, diffraction peaks at 19° and 23° in the XRD pattern and a mass loss of up to 37.3% starting from over 200° C. on thermogravimetric curves, confirmed the attachment of BP-PEG.

Dynamic light scattering (DLS) experiments demonstrated that the NPIAs were highly dispersed in water after surface modification, with hydrodynamic diameters (Dₕ) maintained at 43.8 nm for PEGylated CoₓFe₃₋ₓO₄@NaYF₄(Yb, Er) NPIAs over a concentration range from 0.01 to 1 g/L. Suspensions of NPs CoₓFe₃₋ₓO₄@NaYF₄(Yb, Er)-BP-PEG were extremely stable and could not be precipitated by centrifugation even at 10,000 rpm (9,400 g) for 30 minutes. No aggregation was observed during a period of 14 hours by DLS. This extreme long-term stability is presumed to be due to the strong coordinative interactions between the bisphosphonate groups and metallic sites (i.e. Y³⁺, Yb³⁺ or Er³⁺) on the surface of NPIAs, in keeping with earlier observations. Surface potential plays an important role in determining the bio-distribution and kinetics of NPIAs. The potential environment of NPIAs was studied by measuring the zeta potential, where CoₓFe₃₋ₓO₄@NaYF₄(Yb, Er)-BP-PEG NPs with a long PEG chain (10 KDa) exhibited a negative zeta potential (ca. −10 mV), whereas Fe₃O₄@NaYF₄(Yb, Tm)-BP-PEG NPIAs with shorter PEG chain (2K Da) had a slightly positive charge (ca. +10 mV). All MSA coated nanoparticles displayed a negative zeta potential which increased in magnitude with the amount of MSA on the surface.

The NaYF₄ shell of NPIAs was chosen in part due to a high affinity for [¹⁸F]-fluoride, as fluoride binding to NaYF₄ has been reported previously. Indeed, ¹⁸F-labelling efficiency of MSA-functionalised CoₓFe₃₋ₓO₄@NaYF₄(Yb, Er)-MSA NPIAs (1 mg) was up to 87.7% after brief (5 min) incubation with aqueous no-carrier-added [¹⁸F]-fluoride at room temperature (FIG. 8(a)). These observations are consistent with the core/shell structure of the NPIAs, since neither CoₓFe₃₋ₓO₄ nor Fe₃O₄ alone show significant binding to [¹⁸F]-fluoride. The organic surface coating (BP-PEG or MSA) adversely affected ¹⁸F adsorption: the more ligands on the surface the less [¹⁸F]-fluoride the particle could adsorb during the same incubation time. After incubating 0.1 mg NPIAs with [¹⁸F]-fluoride solution for 5 minutes, CoₓFe₃₋ₓO₄@NaYF₄(Yb, Er)-BP-PEG NPIAs containing 37.5% PEG 37.5% showed a labelling efficiency of 38.5%, lower than 60.1% for CoₓFe₃₋ₓO₄@NaYF₄(Yb, Er)-MSA NPIAs containing 18% MSA. In addition, the labelling efficiency (% of radionuclide bound) was found to increase with the amount of NPIAs, which is consistent with previous observations. Serum stability of [¹⁸F]-fluoride pre-labelled CoₓFe₃₋ₓO₄@NaYF₄(Yb, Er)-BP-PEG NPIAs was determined by incubating the fluorinated particles in human serum for intervals of up to 2 hours and measuring the fraction of activity remaining bound to NPIAs with a gamma counter after separating NPIAs from the supernatant by a NanoSep device with a cut-off size of 10K. As shown in FIG. 5, over 85% of the ¹⁸F remained bound to the NPIAs after incubation in serum up to 2 hours, slightly less than ca. 90% reported in PBS (phosphate buffered saline). The initial partial release of ¹⁸F from NPIAs into serum seemed to be a rapid process since no further changes was observed after 15 minutes, suggesting two modes of binding, one labile (15%) and one inert (85%).

Because of the previously observed strong interactions between bisphosphonate-PEG conjugates and CoₓFe₃₋ₓO₄@NaYF₄(Yb, Er) NPIAs, it was expected that the NPIAs would have a high affinity for radiometal chelate-bisphosphonate conjugates. Labelling with ^{99m}Tc-MDP (in which the bisphosphonate group is bound to Tc) and ^{99m}Tc-DPA-ale (in which the bisphosphonate group is uncoordinated) was carried out separately on MSA functionalised NPIAs. The labelling efficiency with ^{99m}Tc-DPA-ale was found to be up to 77.9% in 1 mg/mL of CoₓFe₃₋ₓO₄@NaYF₄(Yb, Er)-MSA, much higher than that of ^{99m}Tc-MDP, as shown in FIGS. 6 and 8a. The significant difference in labelling between these two forms of ^{99m}Tc could be attributed to their structure; the bisphosphonate group within ^{99m}Tc-DPA-ale is uncoordinated and available for binding to NPIAs whereas the bisphosphonate group within ^{99m}Tc-MDP is coordinated to Tc, which presumably compromises its ability to bind to the NPIA surface. Compared to the ^{99m}Tc-bisphosphonate complexes, all particles showed a higher affinity to ⁶⁴Cu bis(dithiocarmabate)bisphosphonate conjugate (⁶⁴Cu-(DTCBP)₂), which has two uncoordinated bisphosphonate groups, with up to 96% labelling efficiency, as shown in FIG. 8a. The ability to bind readily with [¹⁸F]-fluoride and bisphosphonate conjugates of ⁶⁴Cu and ^{99m}Tc offers potential applications in PET and SPECT imaging.

The r₁ and r₂ relaxivities were measured in aqueous solution at magnetic fields of 3 T and 7 T, to determine the feasibility of using these core/shell structures as MRI contrast. FIG. 8c shows the T₁, T₂ and T₂* weighted MR images of aqueous solutions containing PEGylated Fe₃O₄@NaYF₄ (Yb, Tm) NPIAs at different concentrations. The value of relaxivities r₁ and r₂ of CoₓFe₃₋ₓO₄@NaYF₄(Yb, Er)-BP-PEG in aqueous solution at 3 T was calculated as 4.97 and 102.3 mM⁻¹s⁻¹ respectively. At a higher magnetic field (7 T), r₁ and r₂ were found to be 1.96 and 158.9 mM⁻¹s⁻¹ respectively, as shown in FIG. 9. Fe₃O₄@NaYF₄(Yb, Tm)-BP-PEG NPIAs showed a r₂ relaxivity of up to 325.9 mM⁻¹s⁻¹, a r₂* value of 365.9 mM⁻¹s⁻¹ and a r₁ value of 2.7 mM⁻¹s⁻¹ at 3 T, as shown in FIGS. 8c and 8d. A high r₂ value and r₂/r₁ ratio for both CoₓFe₃₋ₓO₄@NaYF₄(Yb, Er)-BP-PEG and Fe₃O₄@NaYF₄(Yb, Tm)-BP-PEG demonstrated their excellent potential as T₂ or T₂* contrast agents in MRI. Indeed Fe₃O₄@NaYF₄(Yb, Tm)-BP-PEG NPIAs, as multimodal contrast, provides a higher r₂ relaxivity than clinically-used Feridex (r₂≈107 mM⁻¹s⁻¹, r₂/r₁≈4.65) and most iron oxide or iron nanoparticle-based single-modality T₂ MRI contrast agents reported so far.

NaYF₄ and its paramagnetic analogue NaGdF₄ have been intensively investigated as host materials, into which rare earth cations can be doped or co-doped to achieve down-conversion or up-conversion fluorescence. In order to incorporate this type of optical activity into our multimodality contrast nanoparticles, lanthanide cations Er, or Tm (active cations) were co-doped with Yb³⁺ (sensitiser) into the NaYF₄ layer. Up-conversion fluorescent emission was then found under excitation with a 980 nm laser, as shown in FIG. 8(b), FIG. 10 and FIG. 11. A dominant emission at 800 nm, corresponding to the transition from ³H₄ to ³H₆, was observed for Fe₃O₄@NaYF₄(Yb, Tm)-BP-PEG NPIAs. Fluorescence can be improved by using a thicker shell of NaYF₄, either by increasing the ratio of NaYF₄ to iron/cobalt during the deposition of the shell, or by a depositing a second layer of NaYF₄ to give, for example, Fe₃O₄@NaYF₄ (Yb, Tm)@NaYF₄. An improved fluorescence (FIG. 11(e)) was observed after deposition of a thicker NaYF₄ layer before PEGylation (FIG. 11(d) compared to (b)).

### Example 2

### Biological Results Using Nanoparticles

To investigate the bio-distribution of these NPIAs after systemic (intravenous) administration, a solution of the PEGylated CoₓFe₃₋ₓO₄@NaYF₄(Yb, Er)-BP-PEG (10K) (130 μL, 5.6 MBq, 40 μg Fe) and Fe₃O₄@NaYF₄(Yb, Tm)-BP-PEG (2K) NPIAs (150 μL, 3.7 MBq, 45 μg Fe) were injected into the tail veins of two different mice, immediately followed by imaging by co registered PET-MRI. In both cases, radioactivity was taken up by the spleen and liver within 60 minutes, and also accumulated in bladder, as shown in FIGS. 12 and 13(a) to (h), which was observed as darkening contrast on MR images as well, as shown in FIGS. 13 (i) to (l). Very little radionuclide accumulation in skeleton was observed, indicating that the NPIA-radiolabel bond was reasonably stable in vivo over a one-hour time period. The later increase in radioactivity in the bladder and bone which coincided with a decrease of radioactivity in the liver suggests that the particles may be degraded in liver with release of free fluoride.

In vivo PET/MRI imaging of the lymph node (LN) system was carried out using a preclinical nanoScan® PET•MRI scanner with 1 T magnetic field (Mediso Ltd, H-1047, Budapest, Hungary), utilising ¹⁸F-labelled CoₓFe₃₋ₓO₄@NaYF₄(Yb, Er)-BP-PEG (10 K) or ¹⁸F-labelled Fe₃O₄@NaYF₄(Yb, Tm)-BP-PEG (2 K) NPIAs as probes. After injection of 20 μL of [¹⁸F]-fluoride-labelled CoₓFe₃₋ₓO₄@NaYF₄(Yb, Er)-BP-PEG solution containing 6.3 MBq radioactivity and 20 μg Fe into the rear right foot pad of a mouse (C57BL/6, female, 6-7 weeks old, 20 g), coregistered PET and MRI images were recorded 6 hours post injection. The lymph nodes (LNs) were clearly visible on PET images, as shown in FIG. 5. The most prominent signal was from the popliteal LN, which is the nearest draining LN from the injection point, and the next most prominent signal was from the medial iliac LN (FIG. 5a, 5c & 5d). Both LNs were evident on the MR image with a decrease in MR T₂ signal intensity compared to the contralateral (control) LNs. Interestingly, a PET signal was also detected at the more distant lumbar aortic LN. However, no contrast was observed at this area on MR image post injection, due to the relatively poor sensitivity of MRI compared to PET, as shown in FIG. 14(e).

To further explore the potential value of these NPIAs, a more detailed lymph node study by PET/MRI imaging was undertaken, investigating the popliteal LN in response to an acute inflammatory stimulus in the foot. A solution of ¹⁸F-labelled Fe₃O₄@NaYF₄(Yb, Tm)-BP-PEG (20 μL, 4.5 MBq, 20 μg Fe) was injected into each of the two rear footpads of a female C57BL/6 mouse in which only the right leg was inflamed. Again, both popliteal and iliac LNs were identified by PET and MRI 6 hours post injection, as shown in FIG. 15. Interestingly, the right popliteal LN appeared on the MR image as a white spot with a darkened background while the left counterpart displayed a black spot with a white background typical of a healthy lymph node, indicating that the NPIAs accumulated inside the left LN but outside or peripheral to the right (inflamed side) LN, as shown in FIG. 21. Thus, while PET has the sensitivity to easily locate the relevant lymph node, MRI provided the resolution lacking in PET to pinpoint its position more precisely and to delineate disease-related and hence potentially diagnostic changes in the fine structure and distribution of contrast agents in and around the LN. A control experiment was carried out with [¹⁸F]-fluoride but without NPs Fe₃O₄@NaYF₄(Yb, Tm)-BP-PEG, under the same conditions. The PET image in this case did not show any signal at the lymph nodes and only the skeleton was seen, as shown in FIG. 15, confirming that the PET signal in the LN of the NPIA-treated mice came from [¹⁸F]-labelled NPs. By MRI scanning prior to injection of the labellend NPs, no contrast difference was seen on MR images between the normal LN and the inflamed one. The popliteal lymph nodes on the inflamed side were dissected and viewed, together with the adjacent fatty tissue, with a Femtonics (Budapest, Hungary) Fem2D in vivo multi-photon laser scanning microscope under excitation of a 980 nm laser. Strong intracellular fluorescence was observed in the NPIA labelled lymph node, as shown in FIG. 16(a), while only a weak auto fluorescence was detected from actin filaments and collagen fibres for the inflamed but unlabelled one, as shown in FIG. 16(b). Fluorescent NPIAs were also found inside cells of adjacent fat tissue of the inflamed popliteal LN.

### Discussion

The inventors have presented a novel type of inorganic core-shell NPIA with in-built magnetic, fluorescent and radiolabelling properties, which show potential as probes for MRI, optical imaging and PET/SPECT imaging. Stealth features to evade the immune system and prevent opsonisation are required in some imaging and therapy applications to reduce the off-target toxicity of NPIAs, prolong their circulation time in the blood pool (where this is desirable) and deliver them to specific sites. PEGylation, using the novel bisphosphonate derivative to anchor the PEG to the NPIA surface, was employed not only to stabilise the particles against aggregation in solution by the steric effect, but also to modify their circulation time. PEGylated ligands with different polymeric chain lengths were introduced on the surface of CoₓFe₃₋ₓO₄@NaYF₄(Yb, Er) and Fe₃O₄@NaYF₄(Yb, Tm) NPIAs, to produce water soluble versions: CoₓFe₃₋ₓO₄@NaYF₄(Yb, Er)-BP-PEG and Fe₃O₄@NaYF₄(Yb, Tm)-BP-PEG. In both cases, PEGylation, small hydrodynamic size (<<100 nm) and low zeta-potential should offer the opportunity to control circulation time and avoid immediate reticulendothelial clearance where this is desired for specific applications. Indeed, the longer chain PEGylation of CoₓFe₃₋ₓO₄@NaYF₄(Yb, Er) (10 K) (M_{w}=10 K, n≈227) results in slightly negative zeta potential (−10 mV) and delayed clearance compared to Fe₃O₄@NaYF₄(Yb, Tm)-BP-PEG (2 K) (M_{w}=2K, n≈45 and zeta potential of +10 mV), although its circulation time is shorter than that reported previously for PEGylated iron oxide. This may be attributable to reduced PEG surface coverage (36.7% vs 61%). The extent of PEGylation and the chain length may therefore be optimised for specific applications. The small particle size combined with surface properties also plays an important role in enhancing lymphatic transport. Small particles (less than 100 nm) are transported and taken up more readily whereas the larger nanoparticles are likely to remain in the injection site. PEGylation can improve the uptake in lymph nodes by reducing the non-specific interaction between particles and proteins of the interstitium.

Convenient incorporation of readily available imaging radioisotopes such as [¹⁸F]-fluoride is important for applications in radionuclide imaging. Because of the short half-life and the need for GMP conditions in the daily production of radiopharmaceuticals at hospital sites, radiolabelling procedures must be as simple as possible and avoid requirement for costly specialist facilities. Inorganic nanoparticulate materials have previously been reported that bind [¹⁸F]-fluoride rapidly with high efficiency under mild conditions. The NaYF₄ shell of the core-shell system can efficiently carry ¹⁸F as well as other radionuclides such as ^{99m}Tc or ⁶⁴Cu BP conjugates and the radiolabelling of these particles is extremely simple, quick and efficient. Release of fluoride in vivo from the NPIAs, allowing uptake in bone, is relatively slow compared to both lymphatic transport and reticulendothelial clearance, allowing imaging of both processes.

The luminescent properties of the co-doped NaYF₄ shell allow the microscopic evaluation of excised nodes, and would provide a potential visual guide during surgery. NPs with stronger fluorescent emissions could be developed by deposition of another co-doped NaYF₄ layer on the core-shell structures to increase the up-conversion efficiency, or insertion of a layer of material with a low refractive index between the iron oxide core and the fluorescent NaYF₄ shell to suppress light absorption by iron oxide. For example, much stronger visible emissions at 530 nm, 550 nm and 660 nm were observed for Er and Yb co-doped core-shell structure, when increasing the rare earth (Y, Yb, Er) to iron cations (Fe and/or Co) from 0.38 to 1.75. A typical emission of Tm cations at 800 nm appeared to be much stronger after slightly increasing the rare earth to iron cations ratio from 0.75 to 1, or in the presence of Ag. The balance between the thickness of the fluorescent and radiolabelling shell of NaYF₄ and the size of the magnetic core of Fe₃O₄ could be adjusted to optimise the optical and magnetic properties, respectively, for applications as multi-modal imaging agents.

Accurate location and identification of lymph nodes and detection of the pathology within them is important for studies of tumour metastasis in humans, including the identification of sentinel lymph nodes during surgery, and in rodent models for the study of immune responses to foreign antigens, transplants and tumours. The in vivo imaging studies reported here, although not matched to a current typical clinical imaging protocol, are relevant to sentinel node imaging in support of cancer surgery and suggest several ways in which combined co-registered MR and PET imaging with a single contrast agent can provide additional information and increased confidence in image interpretation. The PET images, by virtue of lower content of irrelevant detail, allow easy identification of regions for closer examination by MRI. In addition, the greater sensitivity of PET allows detection of relevant lymph nodes distant from the disease site (e.g. FIG. 4c-e) which is unlikely to be detected by MRI alone. The accurate pre-surgical location of iliac and popliteal LNs in context of the anatomy of the mouse was achieved only when the PET and MRI images were coregistered and overlaid. The additional anatomical and functional detail permitted by contrast MRI (e.g. the differences in structure and contrast agent distribution in and around the lymph node between left and right popliteal nodes) in regions initially identified by PET, but which the limited resolution of PET cannot show, have the potential to provide useful diagnostic information beyond simply identifying the location of the sentinel node, in advance of surgery. The fluorescence should enable further visual observation of these anatomical and functional changes during surgery and subsequently during pathological examination of excised nodes. Data from each of these imaging modalities can be combined with reassurance that the signal comes from the same contrast agent, and hence the same biodistribution, in each modality.

### Example 3

### Aluminium Hydroxide Stabilised MFe₂O₄ (M=Mn, Fe, or Co)

Magnetic nanoparticles (NPs) MFe₂O₄ (M=Mn, Fe or Co) were stabilised by depositing a Al(OH)₃ layer on the surface by a hydrolysis process. The NPIAs displayed excellent colloidal stability in water and a high affinity to [¹⁸F]-fluoride. The properties of the agents, such as the hydrodynamic size, zeta potential, potential for radiolabelling, and MRI relaxivities were strongly dependant on the thickness and hardness of Al(OH)₃ layer.

The inventors report a novel but simple approach to stabilise magnetic NPs by coating them with an Al(OH)₃ layer. These aluminium hydroxide-coated NPIAs displayed excellent colloidal stability in water. The high affinity between fluoride and Al offered a high labelling efficiency achieved by simply incubating NPIAs with [¹⁸F]-fluoride solution for 5 minutes, yielding materials which have potential applications as dual-modality contrast agents for MRI/PET, radiotherapy, hyperthermia, cell tracking and vaccine adjuvants.

### Example 4

### Results with Aluminium Hydroxide Stabilised MFe₂O₄ (M=Mn, Fe, or Co)

Magnetic NPs MFe₂O₄ (M=Mn, Fe, or Co) were obtained following a thermolysis method reported in the literature. Typically, MnFe₂O₄@Al(OH)₃ or CoFe₂O₄@Al(OH)₃ NPIAs were obtained by adding a 5 mL diethylether (Et₂O) solution containing 1 mmol AlCl₃ to a 100 ml Et₂O solution of 80 mg MnFe₂O₄ NPs (ca. 0.33 mmol) whilst stirring. After 10 minutes, the black mixture was added to 500 μL of water and stirred for a further hour. The NPIAs were precipitated out by the addition of 10 mL acetone, and then isolated by centrifugation, washed with ethanol and re-dispersed in water. Fe₃O₄@Al(OH)₃ samples were also obtained via an alternative quick hydrolysis process, where no water was added prior to the addition of acetone and AlCl₃ was hydrolysed when NPs were being dispersed in water, rather than by a small amount of water in Et₂O. The amount of Al(OH)₃ on NPs was controllable by altering the ratio of Fe₃O₄ NPs to AlCl₃. Transmission electron microscopy (TEM), however, revealed no obvious difference size or morphology before and after coating with Al(OH)₃, as shown in FIGS. 17(a) and (b). This could be attributed to an amorphous nature (or poorly crystallised) shell, as shown in FIG. 17(c). Two weak peaks around 21° in the X-ray diffraction (XRD) pattern appeared after coating and were associated with the nordstrandite phase of Al(OH)₃. The infrared spectrum showed the disappearance of absorption peaks of C—H at 2845 cm⁻¹ and 2950 cm⁻¹ after coating with Al(OH)₃, and the appearance of three absorption peaks at 842 cm⁻¹ and 1645 cm⁻¹ and a broad band from 3000-3500 cm⁻¹, corresponding respectively to the Al—O stretching, the deformation vibration of water, and O—H stretching mode. Nanoparticulate MnFe₂O₄ is soluble in hexane but insoluble in water due to the organic layer (oleylamine and oleic acid) on the surface. Once coated with Al(OH)₃, the NPs become soluble in water but insoluble in hexane, as shown in FIG. 17(d). All these features suggest a coating of Al(OH)₃ replacing the oleylamine on the iron oxide nanoparticles.

X-ray photoelectron spectroscopy (XPS) spectrum and inductively coupled plasma mass spectrometry (ICP-MS) were employed for the compositional studies of Al(OH)₃ coated NPs, both of which indicated that the content of Al increased with the initial reactant ratio of AlCl₃ to magnetic NPs. NPIAs with insufficient Al(OH)₃, for example Fe₃O₄@Al(OH)₃ (1:1, precursors ratio of NPs to AlCl₃), tends to aggregate strongly in water, indicated by TEM images and a large hydrodynamic size (hydrodynamic diameter, Dₕ) of up to 400 nm measured by dynamic light scattering (DLS) experiments. This suggested the important role of Al(OH)₃ in stabilising iron oxide NPs in water by converting the hydrophobic surface of Fe₃O₄ NPs into a hydrophilic version, as well as offering a highly positive surface potential to protect them from aggregation. DLS experiments confirmed that Fe₃O₄@Al(OH)₃ (1:2) NPIAs exhibited a highly positive zeta potential up to +70 mV, and an ultra-small Dₕ of 21 nm, reducing from 43.8 nm for Fe₃O₄ in hexane. These coated NPs appeared to be stable in water with no obvious changes on Dₕ for over 12 months.

Another benefit of an Al(OH)₃ coating is the high affinity to fluoride ions, as Al³⁺ cations have the strongest interaction with F⁻ anions of all metal cations; many Al compounds are well-known as good absorbents to remove fluoride anions in water. Indeed, both MnFe₂O₄@Al(OH)₃ and CoFe₂O₄@Al(OH)₃ NPIAs exhibited a high labelling efficiency (LE) with no-carrier-added ¹⁸F-fluoride of up to 97% for as little as 10 μg NPs, as shown in FIG. 18. The absorption ability of Al(OH)₃-coated NPs was further confirmed by a fluoride selective electrode, using cold NaF instead of tracer level radioactive ¹⁸F, and measured to be up to 44.45 mg (fluoride)/g (NPIAs) for MnFe₂O₄@Al(OH)₃ (10 times higher than 4-7 mg/g of hydroxyapatite). This high affinity to ¹⁸F, combined with the particle's magnetic core offers potential applications in cancer therapy by combined radiotherapy and hyperthermia, which may kill tumours more efficiently. The stability of ¹⁸F on NPIAs was investigated in water and in serum. The results demonstrated that over 99.8% ¹⁸F remained on the NPIAs even after washing with water three times, as shown in FIG. 18(b). However, the stability appeared to becoming worse in the case of 0.08 mg NPIAs, indicating a possibility of unstable binding between NPIAs and ¹⁸F-fluoride anions if the amount of NPIAs is insufficient. Studies on the dynamic stability in human serum indicated that there was a slow release of ¹⁸F from radiolabeled NPIAs (MnFe₂O₄@Al(OH)₃ or CoFe₂O₄@Al(OH)₃) over the period of 4 hours, with ca. 40% ¹⁸F remaining on NPIAs after 4 hours incubation and no obvious further release of ¹⁸F-fluoride was observed afterwards. The release of ¹⁸F into serum could be a combination of processes such as the dissociation of loosely bonded ¹⁸F on the surface, the substitution by other anions in serum, interacting with proteins in serum via hydrogen bonding or ion pairing, and the dissolution of NPIAs itself. In the inventors' case, the slow release of ¹⁸F in serum as opposed to the stability in water could be due to the fact that Al(OH)₃ is sensitive to pH and is readily dissolved in an acidic or alkaline environment.

Interestingly, initial results suggested that Fe₃O₄@Al(OH)₃ samples are much less efficient in radiolabelling of ¹⁸F, than their analogous MnFe₂O₄@Al(OH)₃ and CoFe₂O₄@Al(OH)₃. Moreover, NPs with a better colloidal stability in water, which are coated with a thicker Al(OH)₃ layer, showed a worse performance with LE less than 10%, for example Fe₃O₄@Al(OH)₃ (1:2) and Fe₃O₄@Al(OH)₃ (1:3). NPIA Fe₃O₄@Al(OH)₃ (1:1) has a thinner shell but seem to be more efficient in ¹⁸F radiolabelling. These phenomena lead to the hypothesis that a quick hydrolysis with large amount of water resulted in an unstable Al(OH)₃ layer on the NPs whereas a slow hydrolysis with small amount of water in Et₂O lead to a stable layer. An external unstable Al(OH)₃ layer would be washed into the supernatant during the separation process. Unfortunately, most absorption of [¹⁸F]-fluoride occurred on the surface of Al(OH)₃ shell. Therefore, a low value of LE was obtained for NPs coated with unstable Al(OH)₃ shell. By monitoring the Al concentration in the supernatant after washing and comparing to the initial solution by ICP-MS, we found that almost half amount of alumina was washed out at the first wash for Fe₃O₄@Al(OH)₃ (1:3) and Fe₃O₄@Al(OH)₃ (1:2) samples which were synthesis by the quick hydrolysis process. The alumina remaining on the NPs appear to be stable since no Al was detected in the supernatant after the second and third wash. Correspondingly, these NPIAs displayed a high affinity to [¹⁸F]-fluoride, after washing, of up to 94.9%. Only trace amounts of Al was detected in the supernatant of MnFe₂O₄@Al(OH)₃ or CoFe₂O₄@Al(OH)₃ (both synthesised via a slow hydrolysis) which suggested a stable layer of Al(OH)₃ consistent with the radiolabelling results above.

As expected, these Al(OH)₃-coated NPs displayed the magnetic properties of the cores to some extent and appeared to be active in MR imaging, showing a darkening contrast on the T₂ or T₂* weighted MR images of the solution of NPIAs as a result of shortening transverse relaxation time of water molecules, as shown in FIG. 19. The transverse relaxivity property (r₂) of NPIAs strongly depends on the thickness of shell, weakening dramatically with the increasing of Al(OH)₃ shell, as it was reported to be proportional to the volume fraction of magnetic materials. Fe₃O₄@Al(OH)₃ samples displayed higher relaxivities (r₁ and r₂) after washing off the unstable layer, as shown in FIG. 19; for example, r₂ was improved from 81.6 to 121.9 mM⁻¹s⁻¹ for Fe₃O₄@Al(OH)₃ (1:2) NPIAs, and from 60.5 to 116.6 mM⁻¹s⁻¹ for Fe₃O₄@Al(OH)₃ (1:3) NPIAs at 3 T magnetic field, as shown in FIG. 19. For the samples with a stable layer such as MnFe₂O₄@Al(OH)₃ and CoFe₂O₄@Al(OH)₃, no obvious improvement was observed on the relaxivity properties after washing.

In vivo PET/MRI imaging showed that intravenously administrated Fe₃O₄@Al(OH)₃ (1:2) NPIAs were up-taken quickly by lung and liver in 15 minutes, resulting a darkening contrast on MR images in corresponding area, in despite of their small hydrodynamic size of 21 nm, as shown in FIG. 20. Subsequently radioactivity was observed in the skeleton presumably due to later release of fluoride. The quick clearance of Fe₃O₄@Al(OH)₃ (1:2) NPIAs by the lung and liver was not unexpected, as the in vivo behaviour is determined not only by their hydrodynamic size but also by surface property (surface chemistry and potential). Generally, intravenously administered NPIAs over 100 nm are readily cleared by the reticuloendothelial system (RES) through opsonisation, whilst small particles (10-100 nm) tend to stay in the blood pool longer. Negatively charged NPIAs were readily cleared by phagocytic cells in the RES, while NPIAs with a positive surface can absorb negative proteins in serum, resulting in aggregation and consequently a large accommodation in the lung. Thus, to achieve stealth features, the Al(OH)₃-coated NPs needed further surface modification to neutralise the surface potential and prevent aggregation although their hydrodynamic size were sufficiently small. In this case, polymers with anion functional group such as bisphosphonate polyethyleneglycol (BP-PEG) would be required to modulate the surface potential of NPIAs, in addition to the removal of unstable Al(OH)₃ layer.

The evolution of zeta potential and Dₕ of iron oxide NPs before and after modification with Al(OH)₃ and BP-PEG. This fact can be explained by the weakening of repulsive forces between particles, reflected by the decrease of zeta potential from +67.3 to +52.5 mV. Once coated with BP-PEG (10K Da), highly positive Fe₃O₄@Al(OH)₃ NPIAs were neutralized to −8.5 mV, and the Dₕ reduce dramatically down to 14.5 nm. This further reduction in Dₕ indicated that steric effect between the polymeric PEG chain can protect NPIAs from aggregation more efficiently than electrostatic repulsive forces.

### Discussion

The inventors have presented a novel but simple approach to convert hydrophobic iron oxide based magnetic NPs into hydrophilic particles stabilised by a Al(OH)₃ shell. A slow hydrolysis of AlCl₃ would deposit a stable Al(OH)₃ layer on the NP surface whilst a quick process would result in a loosely bounded shell. MFe₂O₄@Al(OH)₃ (M=Mn, Fe, or Co) NPIAs obtained by either of two methods displayed excellent colloidal stability in water, with a high efficiency for [¹⁸F]-fluoride radiolabelling easily achieved after a minutes incubation. It was possible to optimise the colloidal stability, the ¹⁸F radiolabelling and relaxivity properties through tailoring the thickness and stability of the shell by either altering the ratio of magnetic NPs to the AlCl₃ precursor or rate of deposition of the shell, or by filtration to remove the loosely bonded Al(OH)₃. The highly positively charged surface could be neutralised by coating the surface with BP-PEG, by which the risk of up-taken by lung and liver is expected to be reduced. Extra polymeric coating secures their colloidal stability in serum or in an environment with a high ionic strength. The features of this system, including high efficiency on ¹⁸F labelling, excellent colloidal stability, small hydrodynamic size, good transverse relaxivity and controllable surface potential, suggest Fe₃O₄@Al(OH)₃ has potential applications as a bimodal contrast agent in PET/MRI imaging, and as adjuvants for vaccines.

### SUMMARY

In summary, the inventors have reported the synthesis and characterisation of a series of Fe₃O₄@NaYF₄ core-shell type NPIAs in which the shell was co-doped with lanthanide cations providing optical imaging capabilities, and could also be radiolabelled with [¹⁸F]-fluoride and radio metal-bisphosphonate conjugates, while, the iron oxide based core provided MR contrast. The particles thus offer trimodal imaging using PET/SPECT, MRI, and up-conversion fluorescent imaging. The NPs showed excellent colloidal stability in water and a narrow size distribution after surface modification with BP-PEG. The major advantages of these materials as PET/SPECT tracers is the simple and quick radiolabelling process, which is essential for routine clinical use. The in and ex vivo studies in lymph nodes demonstrated the potential advantages of combining imaging modalities using NPIAs as multi-modal (PET, MRI and optical) imaging agents. In addition, these nanoparticles could also potentially acts as visual guides during surgery due to their up-conversion fluorescent properties.

Here the inventors report multi-modal nanoparticulate materials offering magnetic, radionuclide and fluorescent imaging capabilities to exploit the complementary advantages of MR, PET/SPECT and optical imaging. CoₓFe₃₋ₓO₄@NaYF₄(Yb, Er) and Fe₃O₄@NaYF₄(Yb, Tm) core/shell NPIAs with a narrow size distribution were synthesised by the thermal decomposition of metal organic precursors and corresponding trifluoroacetate salts. The CoₓFe₃₋ₓO₄ and Fe₃O₄ magnetic cores provided efficient MRI T₂ contrast with r₂ relaxivity up to 325.9 mM⁻¹s⁻¹ (calculated on basis of Fe concentration) at 3 T, while the NaYF₄ shell could conveniently be radiolabelled with [¹⁸F]-fluoride or radiometal-bisphosphonate conjugates (e.g. ⁶⁴Cu and ^{99m}Tc) by simply incubating an aqueous particle suspension with the chosen radiotracer. The NaYF₄ shell offered fluorescence imaging with emissions in the near infrared region from 500 to 800 nm under excitation at 980 nm, by co-doping with Yb and Er, or Tm. Electron microscopy images showed a narrow size distribution with a mean diameter of 12.2±1.7 nm for CoₓFe₃₋ₓO₄@NaYF₄(Yb, Er) and 10.9±1.5 nm for Fe₃O₄@NaYF₄(Yb, Tm). Dynamic light scattering (DLS) results suggested that these NPIAs can be stabilised by bisphosphonate polyethylene glycol conjugates (BP-PEG), giving a hydrodynamic diameter of 43.8 nm for CoₓFe₃₋ₓO₄@NaYF₄(Yb, Er)-BP-PEG (10K).

The feasibility and potential advantages of using these NPIAs for sentinel lymph node imaging in vivo were demonstrated in mice with CoₓFe₃₋ₓO₄@NaYF₄(Yb, Er)-BP-PEG (average molecular weight M_{w} of PEG chain=10,000, average number of repeat unit n≈227) and Fe₃O₄@NaYF₄(Yb, Tm)-BP-PEG (M_{w}=2000, n≈45) as multi-modality contrast agents. The bio-distribution of intravenously administered particles determined by PET/MR imaging suggested that negatively charged CoₓFe₃₋ₓO₄@NaYF₄(Yb, Er)-BP-PEG (10K) NPIAs stayed in the blood pool longer than positively charged NPs Fe₃O₄@NaYF₄(Yb, Tm)-BP-PEG (2K).

1. A nanoparticle imaging agent comprising an inner magnetic core, and an outer shell disposed substantially around the core, wherein the shell is configured to be radiolabelled.

2. A nanoparticle imaging agent according to claim 1, wherein the magnetic core is a paramagnetic or superparamagnetic material.

3. A nanoparticle imaging agent according to claim 1, wherein the magnetic core comprises iron, nickel, cobalt or dysprosium or a compound, such as an oxide or alloy, which contains one or more of these elements.

4. A nanoparticle imaging agent according to claim 1, wherein the magnetic core comprises magnetite (Fe₃O₄).

5. A nanoparticle imaging agent according to claim 1, wherein the magnetic core comprises MFe₂O₄, wherein M is Mn, Fe or Co.

6. A nanoparticle imaging agent according to claim 1, wherein the outer shell comprises a material that can be radiolabelled.

7. A nanoparticle imaging agent according to claim 1, wherein the outer shell comprises a biocompatible material that has a high affinity for fluoride.

8. A nanoparticle imaging agent according to claim 1, wherein the outer shell comprises NaYF₄ or Al(OH)₃.

9. A nanoparticle imaging agent according to claim 1, wherein the outer shell is attached to the magnetic core by physical absorption, by covalent bonding and/or by epitaxial growth.

10. A nanoparticle imaging agent according to claim 1, wherein the amount of shell attached to the magnetic core is enough so that the outer shell is disposed substantially around the core.

11. A nanoparticle imaging agent according to claim 1, wherein the nanoparticle imaging agent comprises, or is doped with, a rare earth metal.

12. A nanoparticle imaging agent according to claim 11, wherein the rare earth metal is fluorescent.

13. A nanoparticle imaging agent according to claim 11, wherein the rare earth metal is a lanthanide.

14. A nanoparticle imaging agent according to claim 11, wherein the rare earth metal is a lanthanide cation, such as ytterbium (Yb), erbium (Er), thulium (Tm) or holmium (Ho) cation.

15. A nanoparticle imaging agent according to claim 11, wherein the outer shell may be doped with at least one, two, three, or four rare earth metal materials.

16. A nanoparticle imaging agent according to claim 11, wherein the nanoparticle imaging agent is co-doped with ytterbium (Yb) and another rare earth metal, such as erbium (Er), thulium (Tm) or holmium (Ho) cations.

17. A nanoparticle imaging agent according to claim 1, wherein the nanoparticle imaging agent comprises a further doped layer to form a second outer shell disposed around the first outer shell disposed around the inner magnetic core, or another layer of low refractive index material between magnetic core and fluorescent layer.

18. A nanoparticle imaging agent according to claim 1, wherein the nanoparticle imaging agent comprises 1, 2, 3, 4 or 5 shells.

19. A nanoparticle imaging agent according to claim 1, wherein the outer shell comprises one or more ligands.

20. A nanoparticle imaging agent according to claim 19, wherein the more than one ligands are arranged in a spaced-apart array covering the outer surface of the outer shell.

21. A nanoparticle imaging agent according to claim 19, wherein the shell is functionalised with one species of ligand.

22. A nanoparticle imaging agent according to claim 19, wherein the shell is functionalised with two or more species of ligand.

23. A nanoparticle imaging agent according to claim 19, wherein the ligand/s is/are attached to the outer shell by strong coordinative interactions between phosphate groups of bisphosphonate (BP) and metallic sites on the particle surface.

24. A nanoparticle imaging agent according to claim 19, wherein the ligand/s comprise a polymer.

25. A nanoparticle imaging agent according to claim 24, wherein the polymer is a polypeptide, a charged protein, a polysaccharide or a nucleic acid.

26. A nanoparticle imaging agent according to claim 24, wherein the polymer is chitosan, collagen, gelatine, hyaluronic acid, poly(ethylene glycol) (PEG), bisphosphonate poly(ethylene glycol) (BP-PEG), poly(lactic acid), poly(glycolic acid), poly(epsilon-caprolactone), or poly(acrylic acid).

27. A method of preparing a nanoparticle imaging agent according to any preceding claim, the method comprising:—
(i) heating a magnetic metal precursor in a solvent to produce a magnetic core;
(ii) depositing a layer substantially around the magnetic core to produce an outer shell, and
(iii) radiolabelling the shell, to produce a nanoparticle imaging agent.

28. Use of a nanoparticle imaging agent according to claim 1, in an imaging technique.

29. Use according to claim 28, where in the imaging technique is PET, PET/SPECT, MRI or fluorescence imaging.

30. A nanoparticle imaging agent according to claim 1, for use in diagnosis.

31. A nanoparticle imaging agent according to claim 1, for use in surgery.

32. Use of a nanoparticle imaging agent according to claim 1, as a biolabel.

33. A biolabel comprising a nanoparticle imaging agent according to claim 1.

34. A nanoparticle imaging agent according to claim 1, for use in therapy, and preferably as a medicament.

35. A nanoparticle imaging agent according to claim 1, for use in treating inflammatory disease, such as atherosclerosis or arthritis, solid tumors, haematological diseases and malignancies and autoimmune diseases.

36. Use of a nanoparticle imaging agent according to claim 1, as an adjuvant for a vaccine.

37. Use according to claim 36, wherein the shell comprises Al(OH)₃.
